SEQUENCE LISTING (278 sequences)

SEQ ID NO: 1 - RNA, 20 nt, Homo sapiens
ugcgagugcgcgcggccgcg

SEQ ID NO: 2 - RNA, 20 nt, Homo sapiens
gcagcgggugcgagugcgcg

SEQ ID NO: 3 - RNA, 20 nt, Homo sapiens
aggagggccucgggggcagc

SEQ ID NO: 4 - RNA, 20 nt, Homo sapiens
caggagggccucgggggcag

SEQ ID NO: 5 - RNA, 20 nt, Homo sapiens
acucgcacccgcugcccccg

SEQ ID NO: 6 - RNA, 20 nt, Homo sapiens
agagugcaggagggccucgg

SEQ ID NO: 7 - RNA, 20 nt, Homo sapiens
gagagugcaggagggccucg

SEQ ID NO: 8 - RNA, 20 nt, Homo sapiens
ggagagugcaggagggccuc

SEQ ID NO: 9 - RNA, 20 nt, Homo sapiens
gggagagugcaggagggccu

SEQ ID NO: 10 - RNA, 20 nt, Homo sapiens
gcgccggggagagugcagga

SEQ ID NO: 11 - RNA, 20 nt, Homo sapiens
ggcgccggggagagugcagg

SEQ ID NO: 12 - RNA, 20 nt, Homo sapiens
agcggcgccggggagagugc

SEQ ID NO: 13 - RNA, 20 nt, Homo sapiens
aggcccuccugcacucuccc

SEQ ID NO: 14 - RNA, 20 nt, Homo sapiens
agggccggagagcggcgccg

SEQ ID NO: 15 - RNA, 20 nt, Homo sapiens
gagggccggagagcggcgcc

SEQ ID NO: 16 - RNA, 20 nt, Homo sapiens
cgagggccggagagcggcgc

SEQ ID NO: 17 - RNA, 20 nt, Homo sapiens
cucuccccggcgccgcucuc

SEQ ID NO: 18 - RNA, 20 nt, Homo sapiens
acagggcgagggccggagag

SEQ ID NO: 19 - RNA, 20 nt, Homo sapiens
gcggcggacagggcgagggc

SEQ ID NO: 20 - RNA, 20 nt, Homo sapiens
gguggcggcggacagggcga

SEQ ID NO: 21 - RNA, 20 nt, Homo sapiens
cgguggcggcggacagggcg

SEQ ID NO: 22 - RNA, 20 nt, Homo sapiens
ggcggcgguggcggcggaca

SEQ ID NO: 23 - RNA, 20 nt, Homo sapiens
cggcggcgguggcggcggac

SEQ ID NO: 24 - RNA, 20 nt, Homo sapiens
ggcggcggcggcgguggcgg

SEQ ID NO: 25 - RNA, 20 nt, Homo sapiens
ucuggcggcggcggcggugg

SEQ ID NO: 26 - RNA, 20 nt, Homo sapiens
gacucuggcggcggcggcgg

SEQ ID NO: 27 - RNA, 20 nt, Homo sapiens
ggcgacucuggcggcggcgg

SEQ ID NO: 28 - RNA, 20 nt, Homo sapiens
cauggcgacucuggcggcgg

SEQ ID NO: 29 - RNA, 20 nt, Homo sapiens
cugcauggcgacucuggcgg

SEQ ID NO: 30 - RNA, 20 nt, Homo sapiens
gaucugcauggcgacucugg

SEQ ID NO: 31 - RNA, 20 nt, Homo sapiens
cgggaucugcauggcgacuc

SEQ ID NO: 32 - RNA, 20 nt, Homo sapiens
agcggcgcgcgggaucugca

SEQ ID NO: 33 - RNA, 20 nt, Homo sapiens
gcgggagaagagcggcgcgc

SEQ ID NO: 34 - RNA, 20 nt, Homo sapiens
agcgggagaagagcggcgcg

SEQ ID NO: 35 - RNA, 20 nt, Homo sapiens
cagcagcagcgggagaagag

SEQ ID NO: 36 - RNA, 20 nt, Homo sapiens
ccagcagcagcagcagcagc

SEQ ID NO: 37 - RNA, 20 nt, Homo sapiens
gccagcagcagcagcagcag

SEQ ID NO: 38 - RNA, 20 nt, Homo sapiens
cccgcugcugcugcugcugc

SEQ ID NO: 39 - RNA, 20 nt, Homo sapiens
gcugcugcugcugcugcugg

SEQ ID NO: 40 - RNA, 20 nt, Homo sapiens
gcugcuggcggcgcccgccu

SEQ ID NO: 41 - RNA, 20 nt, Homo sapiens
cgggacagcugcgccgaggc

SEQ ID NO: 42 - RNA, 20 nt, Homo sapiens
ccgggacagcugcgccgagg

SEQ ID NO: 43 - RNA, 20 nt, Homo sapiens
ggcccgggacagcugcgccg

SEQ ID NO: 44 - RNA, 20 nt, Homo sapiens
ccgccucggcgcagcugucc

SEQ ID NO: 45 - RNA, 20 nt, Homo sapiens
cgccucggcgcagcuguccc

SEQ ID NO: 46 - RNA, 20 nt, Homo sapiens
ucggcgcagcugucccgggc

SEQ ID NO: 47 - RNA, 20 nt, Homo sapiens
aggcgccgagcggccggccc

SEQ ID NO: 48 - RNA, 20 nt, Homo sapiens
aaggcgccgagcggccggcc

SEQ ID NO: 49 - RNA, 20 nt, Homo sapiens
gcugucccgggccggccgcu

SEQ ID NO: 50 - RNA, 20 nt, Homo sapiens
ggccaaaggcgccgagcggc

SEQ ID NO: 51 - RNA, 20 nt, Homo sapiens
cggcggccaaaggcgccgag

SEQ ID NO: 52 - RNA, 20 nt, Homo sapiens
ggccggccgcucggcgccuu

SEQ ID NO: 53 - RNA, 20 nt, Homo sapiens
ucugggcacccggcggccaa

SEQ ID NO: 54 - RNA, 20 nt, Homo sapiens
cgcucggcgccuuuggccgc

SEQ ID NO: 55 - RNA, 20 nt, Homo sapiens
gcucggcgccuuuggccgcc

SEQ ID NO: 56 - RNA, 20 nt, Homo sapiens
gcagcggucugggcacccgg

SEQ ID NO: 57 - RNA, 20 nt, Homo sapiens
cucgcagcggucugggcacc

SEQ ID NO: 58 - RNA, 20 nt, Homo sapiens
gcgccggcucgcagcggucu

SEQ ID NO: 59 - RNA, 20 nt, Homo sapiens
cgcgccggcucgcagcgguc

SEQ ID NO: 60 - RNA, 20 nt, Homo sapiens
ggcagcgcgccggcucgcag

SEQ ID NO: 61 - RNA, 20 nt, Homo sapiens
gugcccagaccgcugcgagc

SEQ ID NO: 62 - RNA, 20 nt, Homo sapiens
ggcugcggcgggcagcgcgc

SEQ ID NO: 63 - RNA, 20 nt, Homo sapiens
cgcagugcuccggcugcggc

SEQ ID NO: 64 - RNA, 20 nt, Homo sapiens
ucgcagugcuccggcugcgg

SEQ ID NO: 65 - RNA, 20 nt, Homo sapiens
ggcgcgcugcccgccgcagc

SEQ ID NO: 66 - RNA, 20 nt, Homo sapiens
cccucgcagugcuccggcug

SEQ ID NO: 67 - RNA, 20 nt, Homo sapiens
cggccgcccucgcagugcuc

SEQ ID NO: 68 - RNA, 20 nt, Homo sapiens
gccgcagccggagcacugcg

SEQ ID NO: 69 - RNA, 20 nt, Homo sapiens
ccgcagccggagcacugcga

SEQ ID NO: 70 - RNA, 20 nt, Homo sapiens
cagccggagcacugcgaggg

SEQ ID NO: 71 - RNA, 20 nt, Homo sapiens
cggagcacugcgagggcggc

SEQ ID NO: 72 - RNA, 20 nt, Homo sapiens
ggagcacugcgagggcggcc

SEQ ID NO: 73 - RNA, 20 nt, Homo sapiens
acugcgagggcggccgggcc

SEQ ID NO: 74 - RNA, 20 nt, Homo sapiens
cugcgagggcggccgggccc

SEQ ID NO: 75 - RNA, 20 nt, Homo sapiens
agccgcacgcgucccgggcc

SEQ ID NO: 76 - RNA, 20 nt, Homo sapiens
gcagcagccgcacgcguccc

SEQ ID NO: 77 - RNA, 20 nt, Homo sapiens
cgcagcagccgcacgcgucc

SEQ ID NO: 78 - RNA, 20 nt, Homo sapiens
ggccgggcccgggacgcgug

SEQ ID NO: 79 - RNA, 20 nt, Homo sapiens
ggacgcgugcggcugcugcg

SEQ ID NO: 80 - RNA, 20 nt, Homo sapiens
ugcggcugcugcgaggugug

SEQ ID NO: 81 - RNA, 20 nt, Homo sapiens
cgaggugugcggcgcgcccg

SEQ ID NO: 82 - RNA, 20 nt, Homo sapiens
gaggugugcggcgcgcccga

SEQ ID NO: 83 - RNA, 20 nt, Homo sapiens
aggccgcacgcggcgcccuc

SEQ ID NO: 84 - RNA, 20 nt, Homo sapiens
caggccgcacgcggcgcccu

SEQ ID NO: 85 - RNA, 20 nt, Homo sapiens
gcgcccgagggcgccgcgug

SEQ ID NO: 86 - RNA, 20 nt, Homo sapiens
gcccuccugcaggccgcacg

SEQ ID NO: 87 - RNA, 20 nt, Homo sapiens
gggcgccgcgugcggccugc

SEQ ID NO: 88 - RNA, 20 nt, Homo sapiens
cgccgcgugcggccugcagg

SEQ ID NO: 89 - RNA, 20 nt, Homo sapiens
gccgcgugcggccugcagga

SEQ ID NO: 90 - RNA, 20 nt, Homo sapiens
cgccgcacgggcccuccugc

SEQ ID NO: 91 - RNA, 20 nt, Homo sapiens
ggccugcaggagggcccgug

SEQ ID NO: 92 - RNA, 20 nt, Homo sapiens
acugcagccccucgccgcac

SEQ ID NO: 93 - RNA, 20 nt, Homo sapiens
cacugcagccccucgccgca

SEQ ID NO: 94 - RNA, 20 nt, Homo sapiens
gcaggagggcccgugcggcg

SEQ ID NO: 95 - RNA, 20 nt, Homo sapiens
caggagggcccgugcggcga

SEQ ID NO: 96 - RNA, 20 nt, Homo sapiens
aggagggcccgugcggcgag

SEQ ID NO: 97 - RNA, 20 nt, Homo sapiens
cggcgaggggcugcagugcg

SEQ ID NO: 98 - RNA, 20 nt, Homo sapiens
cugcagugcguggugcccuu

SEQ ID NO: 99 - RNA, 20 nt, Homo sapiens
ugcagugcguggugcccuuc

SEQ ID NO: 100 - RNA, 20 nt, Homo sapiens
gcagugcguggugcccuucg

SEQ ID NO: 101 - RNA, 20 nt, Homo sapiens
gccgaggcuggcaccccgaa

SEQ ID NO: 102 - RNA, 20 nt, Homo sapiens
ggccgaggcuggcaccccga

SEQ ID NO: 103 - RNA, 20 nt, Homo sapiens
gcccuucggggugccagccu

SEQ ID NO: 104 - RNA, 20 nt, Homo sapiens
cgccgcaccguggccgaggc

SEQ ID NO: 105 - RNA, 20 nt, Homo sapiens
cggggugccagccucggcca

SEQ ID NO: 106 - RNA, 20 nt, Homo sapiens
gcgccgccgcaccguggccg

SEQ ID NO: 107 - RNA, 20 nt, Homo sapiens
ugccagccucggccacggug

SEQ ID NO: 108 - RNA, 20 nt, Homo sapiens
cugcgcgcgccgccgcaccg

SEQ ID NO: 109 - RNA, 20 nt, Homo sapiens
cagccucggccacggugcgg

SEQ ID NO: 110 - RNA, 20 nt, Homo sapiens
cacggugcggcggcgcgcgc

SEQ ID NO: 111 - RNA, 20 nt, Homo sapiens
gugcggcggcgcgcgcaggc

SEQ ID NO: 112 - RNA, 20 nt, Homo sapiens
gcuggcgcacacacagaggc

SEQ ID NO: 113 - RNA, 20 nt, Homo sapiens
cgcugcuggcgcacacacag

SEQ ID NO: 114 - RNA, 20 nt, Homo sapiens
gccgcacaccggcucgcugc

SEQ ID NO: 115 - RNA, 20 nt, Homo sapiens
ugugugcgccagcagcgagc

SEQ ID NO: 116 - RNA, 20 nt, Homo sapiens
gccagcagcgagccggugug

SEQ ID NO: 117 - RNA, 20 nt, Homo sapiens
uuggcgucgcugccgcacac

SEQ ID NO: 118 - RNA, 20 nt, Homo sapiens
gcacagguuggcguaggugu

SEQ ID NO: 119 - RNA, 20 nt, Homo sapiens
cagcuggcacagguuggcgu

SEQ ID NO: 120 - RNA, 20 nt, Homo sapiens
ggcgcgcagcuggcacaggu

SEQ ID NO: 121 - RNA, 20 nt, Homo sapiens
uggcggcgcgcagcuggcac

SEQ ID NO: 122 - RNA, 20 nt, Homo sapiens
ggcggcuggcggcgcgcagc

SEQ ID NO: 123 - RNA, 20 nt, Homo sapiens
ccucucggagcggcggcugg

SEQ ID NO: 124 - RNA, 20 nt, Homo sapiens
cagccucucggagcggcggc

SEQ ID NO: 125 - RNA, 20 nt, Homo sapiens
ggugcagccucucggagcgg

SEQ ID NO: 126 - RNA, 20 nt, Homo sapiens
gccggugcagccucucggag

SEQ ID NO: 127 - RNA, 20 nt, Homo sapiens
ccgccagccgccgcuccgag

SEQ ID NO: 128 - RNA, 20 nt, Homo sapiens
cggcggccggugcagccucu

SEQ ID NO: 129 - RNA, 20 nt, Homo sapiens
gccgcuccgagaggcugcac

SEQ ID NO: 130 - RNA, 20 nt, Homo sapiens
cgagaggcugcaccggccgc

SEQ ID NO: 131 - RNA, 20 nt, Homo sapiens
gcaggacgaugaccggcggc

SEQ ID NO: 132 - RNA, 20 nt, Homo sapiens
cgcugcaggacgaugaccgg

SEQ ID NO: 133 - RNA, 20 nt, Homo sapiens
ccgcgcugcaggacgaugac

SEQ ID NO: 134 - RNA, 20 nt, Homo sapiens
ccggucaucguccugcagcg

SEQ ID NO: 135 - RNA, 20 nt, Homo sapiens
ggccgcaggcuccgcgcugc

SEQ ID NO: 136 - RNA, 20 nt, Homo sapiens
guccugcagcgcggagccug

SEQ ID NO: 137 - RNA, 20 nt, Homo sapiens
aucuuccugcccuuggccgc

SEQ ID NO: 138 - RNA, 20 nt, Homo sapiens
cagcgcggagccugcggcca

SEQ ID NO: 139 - RNA, 20 nt, Homo sapiens
agcgcggagccugcggccaa

SEQ ID NO: 140 - RNA, 20 nt, Homo sapiens
cggagccugcggccaagggc

SEQ ID NO: 141 - RNA, 20 nt, Homo sapiens
uguugggaucuuccugcccu

SEQ ID NO: 142 - RNA, 20 nt, Homo sapiens
uauuuauggcgcaaacuguu

SEQ ID NO: 143 - RNA, 20 nt, Homo sapiens
auauuuauggcgcaaacugu

SEQ ID NO: 144 - RNA, 20 nt, Homo sapiens
ccgcgauaaaguuauauuua

SEQ ID NO: 145 - RNA, 20 nt, Homo sapiens
ccauaaauauaacuuuaucg

SEQ ID NO: 146 - RNA, 20 nt, Homo sapiens
auauaacuuuaucgcggacg

SEQ ID NO: 147 - RNA, 20 nt, Homo sapiens
uaacuuuaucgcggacgugg

SEQ ID NO: 148 - RNA, 20 nt, Homo sapiens
ggagaagaucgccccugccg

SEQ ID NO: 149 - RNA, 20 nt, Homo sapiens
uucgauaugaaccacggcag

SEQ ID NO: 150 - RNA, 20 nt, Homo sapiens
auucgauaugaaccacggca

SEQ ID NO: 151 - RNA, 20 nt, Homo sapiens
aauucgauaugaaccacggc

SEQ ID NO: 152 - RNA, 20 nt, Homo sapiens
aaacaauucgauaugaacca

SEQ ID NO: 153 - RNA, 20 nt, Homo sapiens
ggcaccucucguuuagaaaa

SEQ ID NO: 154 - RNA, 20 nt, Homo sapiens
gcuuccguuuucuaaacgag

SEQ ID NO: 155 - RNA, 20 nt, Homo sapiens
guuuucuaaacgagaggugc

SEQ ID NO: 156 - RNA, 20 nt, Homo sapiens
uucuaaacgagaggugccgg

SEQ ID NO: 157 - RNA, 20 nt, Homo sapiens
aacccagacccacuagccac

SEQ ID NO: 158 - RNA, 20 nt, Homo sapiens
cgagaggugccgguggcuag

SEQ ID NO: 159 - RNA, 20 nt, Homo sapiens
gagaggugccgguggcuagu

SEQ ID NO: 160 - RNA, 20 nt, Homo sapiens
gugccgguggcuaguggguc

SEQ ID NO: 161 - RNA, 20 nt, Homo sapiens
ugccgguggcuagugggucu

SEQ ID NO: 162 - RNA, 20 nt, Homo sapiens
ugggucuggguuuauugugu

SEQ ID NO: 163 - RNA, 20 nt, Homo sapiens
ggguuuauugugucggaaga

SEQ ID NO: 164 - RNA, 20 nt, Homo sapiens
gaucgugacaaaugcccacg

SEQ ID NO: 165 - RNA, 20 nt, Homo sapiens
gugcuuguuggucaccacgu

SEQ ID NO: 166 - RNA, 20 nt, Homo sapiens
ggugcuuguuggucaccacg

SEQ ID NO: 167 - RNA, 20 nt, Homo sapiens
aacuuugacccggugcuugu

SEQ ID NO: 168 - RNA, 20 nt, Homo sapiens
acguggugaccaacaagcac

SEQ ID NO: 169 - RNA, 20 nt, Homo sapiens
cguggugaccaacaagcacc

SEQ ID NO: 170 - RNA, 20 nt, Homo sapiens
ucuucagcucaacuuugacc

SEQ ID NO: 171 - RNA, 20 nt, Homo sapiens
gucaaaguugagcugaagaa

SEQ ID NO: 172 - RNA, 20 nt, Homo sapiens
cuugauuuuggcuucguaag

SEQ ID NO: 173 - RNA, 20 nt, Homo sapiens
cacuuacgaagccaaaauca

SEQ ID NO: 174 - RNA, 20 nt, Homo sapiens
cucauccacauccuugauuu

SEQ ID NO: 175 - RNA, 20 nt, Homo sapiens
cgaagccaaaaucaaggaug

SEQ ID NO: 176 - RNA, 20 nt, Homo sapiens
acucaucaaaauugaccacc

SEQ ID NO: 177 - RNA, 20 nt, Homo sapiens
cucaucaaaauugaccacca

SEQ ID NO: 178 - RNA, 20 nt, Homo sapiens
ggacaggcagcuugcccugg

SEQ ID NO: 179 - RNA, 20 nt, Homo sapiens
gcaggacaggcagcuugccc

SEQ ID NO: 180 - RNA, 20 nt, Homo sapiens
gagcggccaagcagcaggac

SEQ ID NO: 181 - RNA, 20 nt, Homo sapiens
aagcugccuguccugcugcu

SEQ ID NO: 182 - RNA, 20 nt, Homo sapiens
cugaggagcggccaagcagc

SEQ ID NO: 183 - RNA, 20 nt, Homo sapiens
ccggccgcagcucugaggag

SEQ ID NO: 184 - RNA, 20 nt, Homo sapiens
cucucccggccgcagcucug

SEQ ID NO: 185 - RNA, 20 nt, Homo sapiens
uuggccgcuccucagagcug

SEQ ID NO: 186 - RNA, 20 nt, Homo sapiens
ccgcuccucagagcugcggc

SEQ ID NO: 187 - RNA, 20 nt, Homo sapiens
cgcuccucagagcugcggcc

SEQ ID NO: 188 - RNA, 20 nt, Homo sapiens
auggcgaccacgaacucucc

SEQ ID NO: 189 - RNA, 20 nt, Homo sapiens
gcugcggccgggagaguucg

SEQ ID NO: 190 - RNA, 20 nt, Homo sapiens
ggagaguucguggucgccau

SEQ ID NO: 191 - RNA, 20 nt, Homo sapiens
aagggaaaacgggcuuccga

SEQ ID NO: 192 - RNA, 20 nt, Homo sapiens
cuguguuuugaagggaaaac

SEQ ID NO: 193 - RNA, 20 nt, Homo sapiens
acuguguuuugaagggaaaa

SEQ ID NO: 194 - RNA, 20 nt, Homo sapiens
gguggugacuguguuuugaa

SEQ ID NO: 195 - RNA, 20 nt, Homo sapiens
cgguggugacuguguuuuga

SEQ ID NO: 196 - RNA, 20 nt, Homo sapiens
cuucaaaacacagucaccac

SEQ ID NO: 197 - RNA, 20 nt, Homo sapiens
uucaaaacacagucaccacc

SEQ ID NO: 198 - RNA, 20 nt, Homo sapiens
gguggugcucacgaucccgg

SEQ ID NO: 199 - RNA, 20 nt, Homo sapiens
cuggguggugcucacgaucc

SEQ ID NO: 200 - RNA, 20 nt, Homo sapiens
cucuuugccgccucgcuggg

SEQ ID NO: 201 - RNA, 20 nt, Homo sapiens
aucgugagcaccacccagcg

SEQ ID NO: 202 - RNA, 20 nt, Homo sapiens
cagcucuuugccgccucgcu

SEQ ID NO: 203 - RNA, 20 nt, Homo sapiens
gugagcaccacccagcgagg

SEQ ID NO: 204 - RNA, 20 nt, Homo sapiens
ccagcucuuugccgccucgc

SEQ ID NO: 205 - RNA, 20 nt, Homo sapiens
ccagcgaggcggcaaagagc

SEQ ID NO: 206 - RNA, 20 nt, Homo sapiens
cagcgaggcggcaaagagcu

SEQ ID NO: 207 - RNA, 20 nt, Homo sapiens
agcgaggcggcaaagagcug

SEQ ID NO: 208 - RNA, 20 nt, Homo sapiens
uguaguccaugucugaguug

SEQ ID NO: 209 - RNA, 20 nt, Homo sapiens
ggggcuccgcaacucagaca

SEQ ID NO: 210 - RNA, 20 nt, Homo sapiens
aguugaugauggcgucgguc

SEQ ID NO: 211 - RNA, 20 nt, Homo sapiens
uccauaguugaugauggcgu

SEQ ID NO: 212 - RNA, 20 nt, Homo sapiens
cgaguuuccauaguugauga

SEQ ID NO: 213 - RNA, 20 nt, Homo sapiens
accgacgccaucaucaacua

SEQ ID NO: 214 - RNA, 20 nt, Homo sapiens
caucaucaacuauggaaacu

SEQ ID NO: 215 - RNA, 20 nt, Homo sapiens
aucaucaacuauggaaacuc

SEQ ID NO: 216 - RNA, 20 nt, Homo sapiens
aucaacuauggaaacucggg

SEQ ID NO: 217 - RNA, 20 nt, Homo sapiens
caccguccagguuuacuaac

SEQ ID NO: 218 - RNA, 20 nt, Homo sapiens
ucaccguccagguuuacuaa

SEQ ID NO: 219 - RNA, 20 nt, Homo sapiens
gggaggcccguuaguaaacc

SEQ ID NO: 220 - RNA, 20 nt, Homo sapiens
ggcccguuaguaaaccugga

SEQ ID NO: 221 - RNA, 20 nt, Homo sapiens
uuccaaucacuucaccgucc

SEQ ID NO: 222 - RNA, 20 nt, Homo sapiens
aaccuggacggugaagugau

SEQ ID NO: 223 - RNA, 20 nt, Homo sapiens
aacacuuugaaagugacagc

SEQ ID NO: 224 - RNA, 20 nt, Homo sapiens
cuuaucagaugggauugcaa

SEQ ID NO: 225 - RNA, 20 nt, Homo sapiens
acuuuuuaaucuuaucagau

SEQ ID NO: 226 - RNA, 20 nt, Homo sapiens
aacuuuuuaaucuuaucaga

SEQ ID NO: 227 - RNA, 20 nt, Homo sapiens
uaagauuaaaaaguuccuca

SEQ ID NO: 228 - RNA, 20 nt, Homo sapiens
gucggucaugggacuccgug

SEQ ID NO: 229 - RNA, 20 nt, Homo sapiens
uccuuuggccugucggucau

SEQ ID NO: 230 - RNA, 20 nt, Homo sapiens
uuccuuuggccugucgguca

SEQ ID NO: 231 - RNA, 20 nt, Homo sapiens
cacggagucccaugaccgac

SEQ ID NO: 232 - RNA, 20 nt, Homo sapiens
uggcuuuuccuuuggccugu

SEQ ID NO: 233 - RNA, 20 nt, Homo sapiens
ucccaugaccgacaggccaa

SEQ ID NO: 234 - RNA, 20 nt, Homo sapiens
cuuggugauggcuuuuccuu

SEQ ID NO: 235 - RNA, 20 nt, Homo sapiens
aauauacuucuucuugguga

SEQ ID NO: 236 - RNA, 20 nt, Homo sapiens
gauaccaauauacuucuucu

SEQ ID NO: 237 - RNA, 20 nt, Homo sapiens
aucaccaagaagaaguauau

SEQ ID NO: 238 - RNA, 20 nt, Homo sapiens
uggacgugagugacaucauu

SEQ ID NO: 239 - RNA, 20 nt, Homo sapiens
cuucagcucuuuggcuuugc

SEQ ID NO: 240 - RNA, 20 nt, Homo sapiens
gugccgguccuucagcucuu

SEQ ID NO: 241 - RNA, 20 nt, Homo sapiens
cagcaaagccaaagagcuga

SEQ ID NO: 242 - RNA, 20 nt, Homo sapiens
aagccaaagagcugaaggac

SEQ ID NO: 243 - RNA, 20 nt, Homo sapiens
aagagcugaaggaccggcac

SEQ ID NO: 244 - RNA, 20 nt, Homo sapiens
agagcugaaggaccggcacc

SEQ ID NO: 245 - RNA, 20 nt, Homo sapiens
cgucugggaagucccggugc

SEQ ID NO: 246 - RNA, 20 nt, Homo sapiens
agaucacgucugggaagucc

SEQ ID NO: 247 - RNA, 20 nt, Homo sapiens
acgcuccugagaucacgucu

SEQ ID NO: 248 - RNA, 20 nt, Homo sapiens
uacgcuccugagaucacguc

SEQ ID NO: 249 - RNA, 20 nt, Homo sapiens
gacuucccagacgugaucuc

SEQ ID NO: 250 - RNA, 20 nt, Homo sapiens
ccagcuucugcugggguauc

SEQ ID NO: 251 - RNA, 20 nt, Homo sapiens
gagaccaccagcuucugcug

SEQ ID NO: 252 - RNA, 20 nt, Homo sapiens
ugagaccaccagcuucugcu

SEQ ID NO: 253 - RNA, 20 nt, Homo sapiens
uugagaccaccagcuucugc

SEQ ID NO: 254 - RNA, 20 nt, Homo sapiens
ccugauaccccagcagaagc

SEQ ID NO: 255 - RNA, 20 nt, Homo sapiens
gauaccccagcagaagcugg

SEQ ID NO: 256 - RNA, 20 nt, Homo sapiens
agcagaagcugguggucuca

SEQ ID NO: 257 - RNA, 20 nt, Homo sapiens
gacgucauaaucagcaucaa

SEQ ID NO: 258 - RNA, 20 nt, Homo sapiens
cagcaucaauggacaguccg

SEQ ID NO: 259 - RNA, 20 nt, Homo sapiens
gacaucauuggcggagacca

SEQ ID NO: 260 - RNA, 20 nt, Homo sapiens
gacgucgcugacaucauugg

SEQ ID NO: 261 - RNA, 20 nt, Homo sapiens
aaugacgucgcugacaucau

SEQ ID NO: 262 - RNA, 20 nt, Homo sapiens
augucagcgacgucauuaaa

SEQ ID NO: 263 - RNA, 20 nt, Homo sapiens
ugucagcgacgucauuaaaa

SEQ ID NO: 264 - RNA, 20 nt, Homo sapiens
aagggaaagcacccugaaca

SEQ ID NO: 265 - RNA, 20 nt, Homo sapiens
ccugcggaccaccauguuca

SEQ ID NO: 266 - RNA, 20 nt, Homo sapiens
cccugcggaccaccauguuc

SEQ ID NO: 267 - RNA, 20 nt, Homo sapiens
ggaaagcacccugaacaugg

SEQ ID NO: 268 - RNA, 20 nt, Homo sapiens
cccugaacauggugguccgc

SEQ ID NO: 269 - RNA, 20 nt, Homo sapiens
ccugaacauggugguccgca

SEQ ID NO: 270 - RNA, 20 nt, Homo sapiens
cugaacauggugguccgcag

SEQ ID NO: 271 - RNA, 20 nt, Homo sapiens
ugauaucuucauuaccccug

SEQ ID NO: 272 - DNA, 2138 bp, Homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa

SEQ ID NO: 273 - protein, 480 aa, Homo sapiens
MQIPRAALLPLLLLLLAAPASAQLSRAGRSAPLAAGCPDRCEPARCPPQPEHCEGGRARDACGCCEVCGAPEGAACGLQEGPCGEGLQCVVPFGVPASATVRRRAQAGLCVCASSEPVCGSDANTYANLCQLRAASRRSERLHRPPVIVLQRGACGQGQEDPNSLRHKYNFIADVVEKIAPAVVHIELFRKLPFSKREVPVASGSGFIVSEDGLIVTNAHVVTNKHRVKVELKNGATYEAKIKDVDEKADIALIKIDHQGKLPVLLLGRSSELRPGEFVVAIGSPFSLQNTVTTGIVSTTQRGGKELGLRNSDMDYIQTDAIINYGNSGGPLVNLDGEVIGINTLKVTAGISFAIPSDKIKKFLTESHDRQAKGKAITKKKYIGIRMMSLTSSKAKELKDRHRDFPDVISGAYIIEVIPDTPAEAGGLKENDVIISINGQSVVSANDVSDVIKRESTLNMVVRRGNEDIMITVIPEEIDP

SEQ ID NO: 274 - RNA, 22 nt, Artificial Sequence
  Description of Artificial Sequence Synthetic oligonucleotide
guuuuagagcuaugcuguuuug

SEQ ID NO: 275 - RNA, 80 nt, Artificial Sequence
  Description of Artificial Sequence Synthetic oligonucleotide
guuuuagagcuagaaauagcaaguuaaaauaaggcuaguccguuaucaacuugaaaaaguggcaccgagucggugcuuuu

SEQ ID NO: 276 - RNA, 6 nt, Unknown
  Description of Unknown polyadenylation signal sequence
aauaaa

SEQ ID NO: 277 - RNA, 6 nt, Unknown
  Description of Unknown polyadenylation signal sequence
uauaaa

SEQ ID NO: 278 - RNA, 6 nt, Unknown
  Description of Unknown polyadenylation signal sequence
akuaaa

(19) United States
(12) Patent Application Publication
(10) Pub. No.: US 2020/0291427 A1
(43) Pub. Date: Sep. 17, 2020

(54) CRISPR-BASED THERAPEUTICS FOR TARGETING HTRA1 AND METHODS OF USE

(71) Applicant: Gemini Therapeutics Inc., Cambridge, MA (US)

(72) Inventors: Walter Strapps, Cambridge, MA (US); James McLaughlin, Cambridge, MA (US)

(21) Appl. No.: 16/685,137

(22) Filed: Nov. 15, 2019

Related U.S. Application Data
(60) Provisional application No. 62/768,541, filed on Nov. 16, 2018.

(51) Int. Cl. C12N 15/86; C12N 9/22; A61P 27/02; C12N 15/10

(52) U.S. Cl. CPC C12N 15/86; C12N 9/22; C12N 2310/20; C12N 15/1024; A61P 27/02

(57) ABSTRACT

The present disclosure provides compositions and methods for treating, preventing, or inhibiting diseases of the eye. In one aspect, the disclosure provides compositions comprising HTRA1 guide RNA sequences and uses thereof.

### CROSS-REFERENCE TO RELATED APPLICATIONS

This application claims the benefit of priority from U.S. Provisional Application No. 62/768,541, filed Nov. 16, 2018. The specification of the foregoing application is incorporated herein by reference in its entirety.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: Early AMD; Intermediate AMD; Advanced non-neovascular (“Dry”) AMD; and Advanced neovascular (“Wet”) AMD. Typically, AMD is identified by the focal hyperpigmentation of the retinal pigment epithelium (RPE) and accumulation of drusen deposits. The size and number of drusen deposits typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040.

There is a need for novel treatments for preventing progression from early to intermediate and/or from intermediate to advanced stages of AMD to prevent loss of vision.

### SUMMARY OF THE DISCLOSURE

In some embodiments, the disclosure provides for a composition comprising a guide RNA and a pharmaceutically acceptable carrier, wherein the guide RNA targets an HTRA1 gene. In some embodiments, the HTRA1 gene encodes a polypeptide comprising an amino acid sequence that is at least 80%, 85%, 90%, 91%, 92%, 93%, 94%, 95%, 96%, 97%, 98%, 99% or 100% identical to the amino acid sequence of SEQ ID NO: 273. In some embodiments, the guide RNA comprises a nucleotide sequence that is at least 100%, 99%, 98%, 97%, 96%, 95%, 94%, 93%, 92%, 91%, 90%, 88%, 85%, or 80° A identical to a sequence selected from SEQ ID NOs: 1-271. In some embodiments, the composition is substantially pyrogen free. In some embodiments, the composition further comprises an RNA-guided DNA binding agent or a nucleic acid encoding an RNA-guided DNA binding agent. In some embodiments, the RNA-guided DNA binding agent is a Cas protein. In some embodiments, the Cas protein is Cas9 or Cpf1. In some embodiments, the Cas protein is Cas9 from Streptococcus pyogenes. In some embodiments, the composition further comprises a trRNA. In some embodiments, the guide RNA further comprises a trRNA. In some embodiments, the guide RNA is in a viral vector. In some embodiments, the viral vector is an AAV vector. In some embodiments, the guide RNA is in a non-viral vector. In some embodiments, the non-viral vector is selected from the group consisting of virosomes, liposomes, immunoliposomes, LNPs, polycation or lipid:nucleic acid conjugates, naked nucleic acid (e.g., naked DNA/RNA), artificial virions. In some embodiments, the guide RNA comprises a 2′-O-methyl (2′-O-Me) modified nucleotide. In some embodiments, the guide RNA comprises a phosphorothioate (PS) bond between nucleotides.

In some embodiments, the disclosure provides for a method of inducing a double-stranded break (DSB) within the HTRA1 gene, comprising delivering a composition to a cell, wherein the composition comprises a guide RNA comprising a guide sequence that targets an HTRA1 gene. In some embodiments, the disclosure provides for a method of modifying the HTRA1 gene comprising delivering a composition to a cell, the method comprising administering to the cell (i) an RNA-guided DNA binding agent or a nucleic acid encoding an RNA-guided DNA binding agent and (ii) a guide RNA comprising a guide sequence that targets an HTRA1 gene. In some embodiments, the disclosure provides for a method of treating a disease or disorder in a subject in need thereof, wherein the disease or disorder is associated with aberrantly expressed HTRA1, wherein the method comprises administering to the subject (i) an RNA-guided DNA binding agent or a nucleic acid encoding an RNA-guided DNA binding agent and (ii) a guide RNA comprising a guide sequence that targets an HTRA1 gene. In some embodiments, the disclosure provides for a method of treating a disease or disorder in a subject in need thereof, wherein HTRA1 is expressed at a level at least 25%, 50%, 75%, 100%, 150%, 200%, 250%, 300%, 350%, 400%, 450%, or 500% greater in the subject having the disease or disorder as compared to the level in a control subject not having the disease or disorder, wherein the method comprises administering to the subject (i) an RNA-guided DNA binding agent or a nucleic acid encoding an RNA-guided DNA binding agent and (ii) a guide RNA comprising a guide sequence that targets an HTRA1 gene. In some embodiments, the disclosure provides for a method of treating age-related macular degeneration in a subject in need thereof, wherein the method comprises administering to the subject (i) an RNA-guided DNA binding agent or a nucleic acid encoding an RNA-guided DNA binding agent and (ii) a guide RNA comprising a guide sequence that targets an HTRA1 gene. In some embodiments, the guide RNA comprises a nucleotide sequence that is at least 100%, 99%, 98%, 97%, 96%, 95%, 94%, 93%, 92%, 91%, 90%, 88%, 85%, or 80% identical to a sequence selected from SEQ ID NOs: 1-271. In some embodiments, the method further comprises inducing a double-stranded break (DSB) within the endogenous HTRAJ gene. In some embodiments, the method further comprises modifying the endogenous HTRAJ gene. In some embodiments, the method further comprises administering a RNA-guided DNA binding agent with the HTRAJ guide RNA. In some embodiments, the guide RNA and RNA-guided DNA binding agent or a nucleic acid encoding an RNA-guided DNA binding agent are administered to the subject in the same composition. In some embodiments, the guide RNA and RNA-guided DNA binding agent or a nucleic acid encoding an RNA-guided DNA binding agent are administered to the subject in separate compositions. In some embodiments, the separate compositions are administered simultaneously. In some embodiments, the separate compositions are administered consecutively. In some embodiments, non-homologous ending joining (NHEJ) leads to a mutation during repair of a DSB in the endogenous HTRAJ gene. In some embodiments, NHEJ leads to a deletion or insertion of a nucleotide(s) during repair of a DSB in the endogenous HTRAJ gene. In some embodiments, the deletion or insertion of a nucleotide(s) induces a frame shift or nonsense mutation in the endogenous HTRAJ gene. In some embodiments, the guide RNA is administered in a nucleic acid vector and/or a lipid nanoparticle. In some embodiments, the RNA-guided DNA binding agent is administered in a nucleic acid vector and/or lipid nanoparticle. In some embodiments, the nucleic acid vector is a viral vector. In some embodiments, the viral vector is selected from the group consisting of an adeno associate viral (AAV) vector, adenovirus vector, retrovirus vector, and lentivirus vector. In some embodiments, the AAV vector is selected from the group consisting of AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof. In some embodiments, the RNA-guided DNA binding agent is a class 2 Cas nuclease. In some embodiments, the Cas nuclease is a Cas9 nuclease. In some embodiments, the Cas9 nuclease is an S. pyogenes Cas9 nuclease. In some embodiments, the Cas nuclease is a cleavase. In some embodiments, the Cas nuclease is a nickase. In some embodiments, the guide RNA is single-stranded or double-stranded. In some embodiments, the nucleic acid construct is a single-stranded DNA or a double-stranded DNA. In some embodiments, the control subject is a subject of the same sex and/or of similar age as the subject having the disease or disorder. In some embodiments, the subject has one or more mutations in the HTRA1 gene. In some embodiments, the one or more mutations are not in the coding sequence for the HTRA1 gene. In some embodiments, the one or more mutations are in 10q26 in a human subject. In some embodiments, the one or more mutations correspond to any one or more of the following polymorphisms in a human subject: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In some embodiments, the subject has age-related macular degeneration. In some embodiments, the subject is a human. In some embodiments, the human is at least 40 years of age. In some embodiments, the human is at least 50 years of age. In some embodiments, the human is at least 65 years of age. In some embodiments, the guide RNA, RNA-guided DNA binding agent and/or nucleic acid encoding the RNA-guided DNA binding agent are administered locally. In some embodiments, the guide RNA, RNA-guided DNA binding agent and/or nucleic acid encoding the RNA-guided DNA binding agent are administered intravitreally. In some embodiments, the guide RNA, RNA-guided DNA binding agent and/or nucleic acid encoding the RNA-guided DNA binding agent are administered subretinally. In some embodiments, the guide RNA, RNA-guided DNA binding agent and/or nucleic acid encoding the RNA-guided DNA binding agent are administered systemically. In some embodiments, the subject has polypoidal choroidal vasculopathy. In some embodiments, the subject has Wet age-related macular degeneration. In some embodiments, the subject has Dry age-related macular degeneration.

### DETAILED DESCRIPTION OF THE DISCLOSURE

In one aspect, the disclosure provides guide RNA compositions that target the HTRA1 gene. Guide RNA sequences targeting the HTRA1 gene include, for example any of the sequences of SEQ ID NOs: 1-271. In some embodiments, the guide RNAs comprising the guide sequences provided herein together with an RNA-guided DNA binding agent (such as a Cas nuclease) induce double-stranded breaks (DSBs) in the HTRA1 gene, and non-homologous ending joining (NHEJ) during repair leads to a mutation in the HTRA1 gene. In some embodiments, NHEJ leads to a deletion or insertion of a nucleotide(s), which induces a frame shift or nonsense mutation in the HTRA1 gene, rendering the gene nonfunctional. In another aspect, the disclosure provides methods of treating, preventing, or inhibiting diseases of the eye by intraocularly (e.g., intravitreally) administering an effective amount of any of the guide RNAs discloses herein with an RNA-guided DNA binding agent (or a polynucleotide encoding an RNA-guided DNA binding agent) such as a Cas nuclease, e.g., Cas9 or mRNA encoding a Cas nuclease, e.g., mRNA encoding Cas9.

A wide variety of diseases of the eye may be treated or prevented using any of the guide RNA compositions and methods provided herein. Diseases of the eye that may be treated or prevented using the compositions and methods of the present disclosure include but are not limited to, glaucoma, macular degeneration (e.g., age-related macular degeneration), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically).

### General Techniques

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR2 (“amidate”), P(O)R, P(O)OR′, CO or CH₂ (“formacetal”), in which each R or R′ is independently H or substituted or unsubstituted alkyl (1-20 C) optionally containing an ether (-O-) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, a “base”, “nucleotide base,” or “nucleobase,” is a heterocyclic pyrimidine or purine compound, which is a standard constituent of all nucleic acids, and includes the bases that form the nucleotides adenine (A), guanine (G), cytosine (C), thymine (T), and uracil (U). A nucleobase may further be modified to include, without limitation, universal bases, hydrophobic bases, promiscuous bases, size-expanded bases, and fluorinated bases. As used herein, the term “nucleotide” can include a modified nucleotide (such as, for example, a nucleotide mimic, abasic residue (Ab), or a surrogate replacement moiety).

As used herein, the terms “sequence” and “nucleotide sequence” mean a succession or order of nucleobases or nucleotides, described with a succession of letters using standard nomenclature.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

In one embodiment, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject is in need of optogenetic therapy. In another embodiment, the subject has shown clinical signs of a disease of the eye.

In some embodiments, the subject has, or is at risk of developing AMD. In some embodiments, the AMD is Early AMD; Intermediate AMD; Advanced non-neovascular (“Dry”) AMD; or Advanced neovascular (“Wet”) AMD.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject for an infection, “prevent”, “preventing” and “prevention” refer to the inhibition or a reduction in the development or onset of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of an infection (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of” a substance, a compound or an agent (e.g., any of the compositions disclosed herein) to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, the term “ocular cells” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In particular embodiments, the cells are RPE cells.

“Guide RNA”, “gRNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” or “guide” refers to each type. The trRNA may be a naturally-occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of a guide RNA for a Streptococcus pyogenes Cas9 (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for Cas proteins include both the positive and negative strands of genomic DNA (i.e., the sequence given and the sequence's reverse compliment), as a nucleic acid substrate for a Cas protein is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. RNA-guided DNA binding agents include Cas proteins (e.g., Cas9 proteins), such as Cas nucleases (e.g., Cas9 nucleases). “Cas nuclease”, also called “Cas protein”, as used herein, encompasses Cas cleavases, Cas nickases, and inactivated forms thereof (“dCas DNA binding agents”). Cas proteins further encompass a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity, such as a Cas9 nuclease or a Cpf1 nuclease. Class 2 Cas nucleases include Class 2 Cas cleavases/nickases (e.g., H840A, D10A, or N863A variants), which further have RNA-guided DNA cleavase or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A/R661A/Q695A/Q926A variants), HypaCas9 (e.g., N692A/M694A/Q695A/H698A variants), eSPCas9(1.0) (e.g, K810A/K1003A/R1060A variants), and eSPCas9(1.1) (e.g., K848A/K1003A/R1060A variants) proteins and modifications thereof. Cpf1 protein, Zetsche et al., Cell, 163: 1-13 (2015), is homologous to Cas9, and contains a RuvC-like nuclease domain. The Cpf1 sequences of Zetsche et al. are incorporated by reference in their entirety. See, e.g., Zetsche et al. at Tables 51 and S3. “Cas9” encompasses Spy Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide that is not DNA and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

As used herein, the term “capable of” means that the referenced composition or method has the capability to perform a specific function, but that it is not required to be performing that specific function at any specific moment in time. The term “capable of” encompasses instances where the composition is actively performing a specific function.

As used herein, “indels” refer to insertion/deletion mutations consisting of a number of nucleotides that are either inserted or deleted at the site of double-stranded breaks (DSBs) in the nucleic acid.

As used herein, “knockdown” or “knocking down” refers to a decrease in expression of a particular gene product (e.g., protein, mRNA, or both). Knockdown of a protein (e.g., HTRA1) can be measured either by detecting protein secreted by tissue or population of cells (e.g., in serum or cell media) or by detecting total cellular amount of the protein from a tissue or cell population of interest. Methods for measuring knockdown of mRNA are known, and include sequencing of mRNA isolated from a tissue or cell population of interest. In some embodiments, “knockdown” may refer to some loss of expression of a particular gene product, for example a decrease in the amount of mRNA transcribed or a decrease in the amount of protein expressed or secreted by a population of cells (including in vivo populations such as those found in tissues).

As used herein, “knockout” or “knocking out” refers to a loss of expression of a particular protein in a cell. Knockout can be measured either by detecting the amount of protein secretion from a tissue or population of cells (e.g., in serum or cell media) or by detecting total cellular amount of a protein a tissue or a population of cells. In some embodiments, the methods of the disclosure “knockout” HTRA1 in one or more cells (e.g., in a population of cells including in vivo populations such as those found in tissues). In some embodiments, a knockout is not the formation of mutant HTRA1 protein, for example, created by indels, but rather the complete loss of expression of HTRA1 protein in a cell.

As used herein, “ribonucleoprotein” (RNP) or “RNP complex” refers to a guide RNA together with an RNA-guided DNA binding agent, such as a Cas protein. In some embodiments, the guide RNA guides an RNA-guided DNA binding agent such as Cas9 to a target sequence, and the guide RNA hybridizes with and an RNA-guided DNA binding agent cleaves the target sequence.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

Each embodiment described herein may be used individually or in combination with any other embodiment described herein.

### Guide RNAs and Modified Guide RNAs Targeting HTRA1

HTRA1 is a serine protease that targets a variety of proteins, including extracellular matrix proteins such as fibronectin. Fibronectin fragments resulting from HTRA1 cleavage are able to further induce synovial cells to up-regulate MMPI and MMP3 production. There is evidence that HTRA1 may also degrade proteoglycans, such as aggrecan, decorin and fibromodulin. By cleaving proteoglycans, HTRA1 may release soluble FGF-glycosaminoglycan complexes that promote the range and intensity of FGF signals in the extracellular space. HTRA1 also regulates the availability of insulin-like growth factors (IGFs) by cleaving IGF-binding proteins. Intracellularly, HTRA1 degrades TSC2, leading to the activation of TSC2 downstream targets.

Overexpression of HTRA1 alters the integrity of Bruch's membrane, which permits choroid capillaries to invade across the extracellular matrix in conditions such as wet age-related macular degeneration. Tong et al., 2010, Mol. Vis., 16:1958-81. HTRA1 also inhibits signaling mediated by TGF-beta family members, which may regulate many physiological processes, including retinal angiogenesis and neuronal survival and maturation during development. It has been previously determined that a single-nucleotide polymorphism (r511200638) in the promoter region of the HTRA1 gene was found to be significantly associated with susceptibility to AMD in various patient populations. Tong et al., 2010.

The subject disclosure provides for compositions that have utility in targeting HTRA1 gene or DNA sequences responsible for regulating an HTRA1 gene. Throughout this disclosure, unless specified otherwise, “HTRA1 gene” will encompass HTRA1 exons, introns and regulatory sequences (e.g., promoters, enhancers, repressor nucleotide sequences).

In some embodiments, any of the compositions disclosed herein comprises one or more guide RNA (gRNA) comprising guide sequences that direct a RNA-guided DNA binding agent (e.g., Cas9) to a target HTRA1 DNA sequence. In some embodiments, the gRNA comprises the nucleotide sequence of any one of SEQ ID NOs: 1-271, or reverse complements thereof. In some embodiments, the gRNA comprises a nucleotide sequence comprising a nucleotide sequence that is at least 80%, 85%, 90%, 93%, 95%, 97%, 98%, 99% or 100% to the nucleotide sequence of any one of SEQ ID NOs: 1-271. In some embodiments, the gRNA comprises a nucleotide sequence of SEQ ID NOs: 1-271, but with at least 1, 2, 3, 4, 5, 6, 7, 8, 9, 10 nucleotide modifications as compared to any one of SEQ ID NOs: 1-271. For example, a gRNA may comprise the nucleotide sequence of SEQ ID NO: 1, but with 2 nucleotide modifications as compared to SEQ ID NO: 1; or the gRNA may comprise the nucleotide sequence of SEQ ID NO: 2, but with 1 nucleotide modification as compared to SEQ ID NO: 2. In some embodiments, any of the gRNA sequences disclosed herein comprises at least 6, 7, 8, 9, 10, 11, 12, 13, 14, 15, 16, 17, 18 or 19 contiguous nucleotides present from a nucleotide sequence that is at least 80%, 85%, 90%, 93%, 95%, 97%, 98%, 99% or 100% identical to any one of SEQ ID NOs: 1-271. Any of the gRNA sequences disclosed herein may further comprise a crRNA and/or a trRNA as known in the art. In each composition and method embodiment described herein, the crRNA and trRNA may be associated on one RNA (sgRNA), or may be on separate RNAs (dgRNA).

In some embodiments, any of the compositions or methods disclosed herein is capable of inhibiting the expression of an HTRA1 protein. In some embodiments, the HTRA1 protein comprises an amino acid sequence that is at least 80%, 85%, 90%, 93%, 95%, 97%, 98%, 99% or 100% identical to SEQ ID NO: 273, or a functional fragment thereof. In some embodiments, any of the compositions or methods disclosed herein is capable of inhibiting the expression of a protein having an amino acid sequence that is at least 80%, 85%, 90%, 93%, 95%, 97%, 98%, 99% or 100% identical to SEQ ID NO: 273, or a functional fragment thereof. In preferred embodiments, any of the compositions or methods disclosed herein target an HTRA1 gene. In some embodiments, the HTRA1 gene may be transcribed into an mRNA transcript, wherein the transcript comprises a nucleotide sequence that is at least 80%, 85%, 90%, 93%, 95%, 97%, 98%, 99% or 100% identical to the nucleotide sequence of SEQ ID NO: 272, but with thymines replaced with uracils, or complements thereof. In some embodiments, any of the compositions or methods disclosed herein is capable of preventing transcription of an mRNA transcript that is at least 80%, 85%, 90%, 93%, 95%, 97%, 98%, 99% or 100% identical to the nucleotide sequence of SEQ ID NO: 272, but with thymines replaced with uracils, or complements thereof. In some embodiments, any of the compositions or methods disclosed herein is capable of inhibiting the expression of HTRA1 protein by at least 5%, 10%, 15%, 25%, 30%, 35%, 40%, 45%, 50%, 55%, 60%, 65%, 70%, 75%, 80%, 85%, 90%, 95% or 100% as compared to the expression level of HTRA1 protein in the absence of the composition or method. In some embodiments, any of the compositions or methods disclosed herein is capable of reducing HTRA1-encoding mRNA levels in a cell. In some embodiments, the composition or method is capable of reducing HTRA1-encoding mRNA levels in a cell by at least 5%, 10%, 15%, 25%, 30%, 35%, 40%, 45%, 50%, 55%, 60%, 65%, 70%, 75%, 80%, 85%, 90%, 95% or 100% as compared to HTRA1-encoding mRNA levels in the same cell type in the absence of the composition or method.

In each of the composition and method embodiments described herein, the guide RNA may comprise two RNA molecules as a “dual guide RNA” or “dgRNA”. The dgRNA comprises a first RNA molecule (e.g. a crRNA) comprising a guide sequence comprising any of the guide sequences disclosed herein, and a second RNA molecule comprising a trRNA. The first and second RNA molecules are not covalently linked, but may form a RNA duplex via the base pairing between portions of the crRNA and the trRNA.

In each of the composition and method embodiments described herein, the guide RNA may comprise a single RNA molecule as a “single guide RNA” or “sgRNA”. The sgRNA comprises a crRNA (or a portion thereof) comprising any one of the guide sequences disclosed herein covalently linked to a trRNA (or a portion thereof). In some embodiments, the crRNA and the trRNA are covalently linked via a linker. In some embodiments, the sgRNA forms a stem-loop structure via the base pairing between portions of the crRNA and the trRNA. In some embodiments, the sgRNA is modified according to the methods described, e.g., in WO2018119182, the contents of which are hereby incorporated by reference in their entirety.

In some embodiments, the trRNA may comprise all or a portion of a wild type trRNA sequence from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In other embodiments, the composition comprises at least two gRNAs comprising guide sequences selected from any two or more of the guide sequences of SEQ ID NOs: 1-271, or fragments thereof. In some embodiments, the composition comprises at least two gRNAs that each are at least 99%, 98%, 97%, 96%, 95%, 94%, 93%, 92%, 91%, or 90% identical to any of the nucleic acids of SEQ ID NOs: 1-271.

In some embodiments, any of the guide sequences disclosed herein may further comprise additional nucleotides to form a crRNA, e.g., with the following exemplary nucleotide sequence following the guide sequence at its 3′ end: GUUUUAGAGCUAUGCUGUUUUG (SEQ ID NO: 274). In the case of a sgRNA, the guide sequences may further comprise additional nucleotides to form a sgRNA, e.g., with the following exemplary nucleotide sequence following the 3′ end of the guide sequence:

GUUUUAGAGCUAGAAAUAGCAAGUUAAAAUAAGGCUAGUCCGUUAUCA
ACUUGAAAAAGUGGCACCGAGUCGGUGCUUUU (SEQ ID NO:
275) in 5′ to 3′ orientation.

The guide RNA compositions described herein are designed to recognize a target sequence in the HTRA1 gene. For example, HTRA1 target sequence may be recognized and cleaved by the provided RNA-guided DNA binding agent (e.g., a Cas nuclease such as Cas9). In some embodiments, a Cas nuclease may be directed by a guide RNA to a target sequence of the HTRA1 gene, where the guide sequence of the guide RNA hybridizes with the target sequence and the Cas nuclease cleaves the target sequence.

In some embodiments, the selection of the one or more guide RNAs is determined based on target sequences within the HTRA1 gene.

Without being bound by any particular theory, mutations in critical regions of the gene may be less tolerable than mutations in non-critical regions of the gene, thus the location of a DSB is an important factor in the amount or type of protein knockdown or knockout that may result. In some embodiments, a guide RNA complementary or having complementarity to a target sequence within HTRA1 is used to direct the Cas nuclease to a particular location in the HTRA1 gene. In some embodiments, guide RNAs are designed to have guide sequences that are complementary or have complementarity to target sequences in exons of HTRA1.

In some embodiments, the present disclosure provides a guide RNA comprising one or more modifications. In some embodiments, the modification comprises a 2′-O-methyl (2′-O-Me) modified nucleotide. In some embodiments, the modification comprises a phosphorothioate (PS) bond between nucleotides.

Modified sugars are believed to control the puckering of nucleotide sugar rings, a physical property that influences oligonucleotide binding affinity for complementary strands, duplex formation, and interaction with nucleases. Substitutions on sugar rings can therefore alter the confirmation and puckering of these sugars. For example, 2′-O-methyl (2′-O-Me) modifications can increase binding affinity and nuclease stability of oligonucleotides, though the effect of any modification at a given position in an oligonucleotide needs to be empirically determined.

The terms “mA,” “mC,” “mU,” or “mG” may be used to denote a nucleotide that has been modified with 2′-O-Me.

Modification of 2′-O-methyl can be depicted as follows:

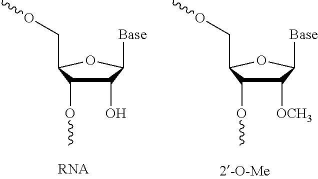

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

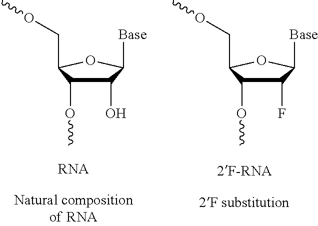

In some embodiments, the modification may be 2′-O-(2-methoxyethyl) (2′-O-moe). Modification of a ribonucleotide as a 2′-O-moe ribonucleotide can be depicted as follows:

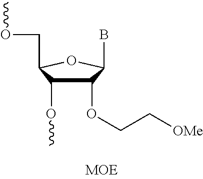

The terms “moeA,” “moeC,” “moeU,” or “moeG” may be used to denote a nucleotide that has been modified with 2′-O-moe.

Phosphorothioate (PS) linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S- into a nonbridging phosphate oxygen, generating a PS bond in lieu of a phosphodiester bond:

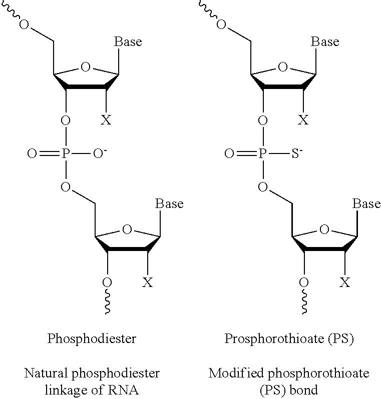

Abasic nucleotides refer to those which lack nitrogenous bases. The figure below depicts an oligonucleotide with an abasic (also known as apurinic) site that lacks a base:

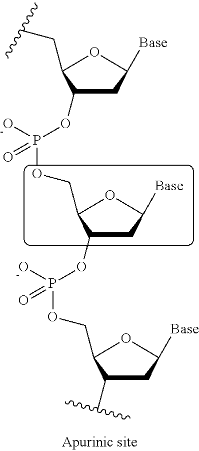

Inverted bases refer to those with linkages that are inverted from the normal 5′ to 3′ linkage (i.e., either a 5′ to 5′ linkage or a 3′ to 3′ linkage). For example:

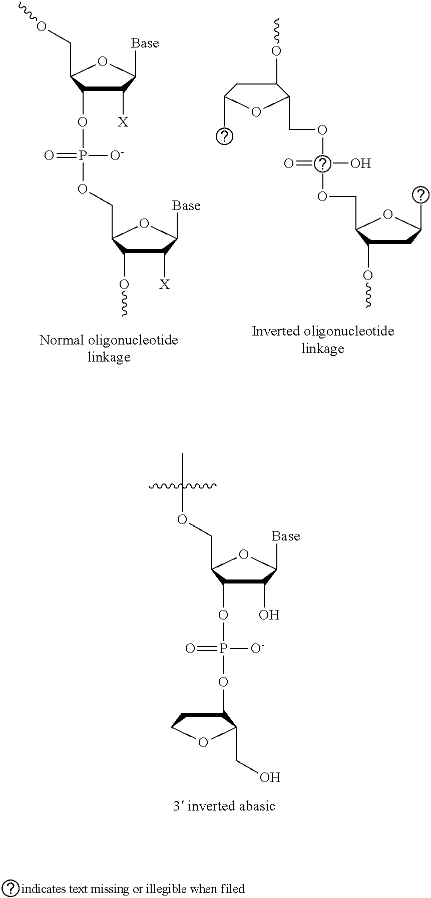

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ end of the 5′ terminus, and one or more of the last three, four, or five nucleotides at the 3′ end of the 3′ terminus are modified. In some embodiments, the modification is a 2′-O-Me, 2′-F, 2′-O-moe, inverted abasic nucleotide, PS bond, or other nucleotide modification well known in the art to increase stability and/or performance.

In some embodiments, the first four nucleotides at the 5′ end of the 5′ terminus, and the last four nucleotides at the 3′ end of the 3′ terminus are linked with phosphorothioate (PS) bonds.

In some embodiments, the first three nucleotides at the 5′ end of the 5′ terminus, and the last three nucleotides at the 3′ end of the 3′ terminus comprise a 2′-O-methyl (2′-O-Me) modified nucleotide. In some embodiments, the first three nucleotides at the 5′ end of the 5′ terminus, and the last three nucleotides at the 3′ end of the 3′ terminus comprise a 2′-fluoro (2′-F) modified nucleotide. In some embodiments, the first three nucleotides at the 5′ end of the 5′ terminus, and the last three nucleotides at the 3′ end of the 3′ terminus comprise an inverted abasic nucleotide.

In some embodiments, the guide RNA comprises a modified sgRNA, as described, e.g., in WO 2018119182, the contents of which are hereby incorporated by reference in their entirety. In some embodiments, the guide RNAs disclosed herein comprise one of the modification pattern disclosed in WO/2018/107028, the contents of which are hereby incorporated by reference in their entirety.

### Ribonucleoprotein Complex

In some embodiments, the present disclosure provides a composition comprising one or more guide RNAs (or any modified form described herein) comprising a: a) guide sequence and b) an RNA-guided DNA binding agent (e.g., Cas9) or a polynucleotide/nucleic acid encoding an RNA-guided DNA binding agent. In some embodiments, the guide sequence is any of the guide sequences disclosed herein (e.g., any of SEQ ID NOs: 1-271). In some embodiments, the guide RNA together with an RNA-guided DNA binding agent such as a Cas9 is called a ribonucleoprotein complex (RNP). In some embodiments, the RNA-guided DNA binding agent is a Cas nuclease. In some embodiments, the guide RNA together with a Cas nuclease is called a Cas RNP. In some embodiments, the RNP comprises Type-I, Type-II, or Type-III components. In some embodiments, the Cas nuclease is from the Type-I CRISPR/Cas system. In some embodiments, the Cas nuclease is from the Type-II CRISPR/Cas system. In some embodiments, the Cas nuclease is from the Type-III CRISPR/Cas system. In some embodiments, the Cas nuclease is Cas9. In some embodiments, the Cas nuclease is Cpf1. In some embodiments, the Cas nuclease is the Cas9 nuclease from the Type-II CRISPR/Cas system. In some embodiment, the guide RNA together with Cas9 is called a Cas9 RNP.

In embodiments encompassing a Cas nuclease, the Cas nuclease may be from a Type-IIA, Type-IIB, or Type-IIC system. Non-limiting exemplary species that the Cas nuclease or other RNP components may be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina. In some embodiments, the Cas nuclease is the Cas9 protein from Streptococcus pyogenes. In some embodiments, the Cas nuclease is the Cas9 protein from Streptococcus thermophilus. In some embodiments, the Cas nuclease is the Cas9 protein from Neisseria meningitidis. In some embodiments, the Cas nuclease is the Cas9 protein is from Staphylococcus aureus. In some embodiments, the Cas nuclease is the Cpf1 protein from Francisella novicida. In some embodiments, the Cas nuclease is the Cpf1 protein from Acidaminococcus sp. In some embodiments, the Cas nuclease is the Cpf1 protein from Lachnospiraceae bacterium ND2006.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition and method embodiments, the Cas induces a double strand break in target DNA.

Modified versions of Cas9 having one catalytic domain, either RuvC or HNH, that is inactive are termed “nickases.” Nickases cut only one strand on the target DNA, thus creating a single-strand break. A single-strand break may also be known as a “nick.” In some embodiments, the compositions and methods comprise nickases. In some embodiments, the compositions and methods comprise a nickase Cas9 that induces a nick rather than a double strand break in the target DNA.

In some embodiments, the Cas protein may be modified to contain only one functional nuclease domain. For example, the Cas protein may be modified such that one of the nuclease domains is mutated or fully or partially deleted to reduce its nucleic acid cleavage activity. In some embodiments, a nickase Cas is used having a RuvC domain with reduced activity. In some embodiments, a nickase Cas is used having an inactive RuvC domain. In some embodiments, a nickase Cas is used having an HNH domain with reduced activity. In some embodiments, a nickase Cas is used having an inactive HNH domain.

In some embodiments, a conserved amino acid within a Cas protein nuclease domain is substituted to reduce or alter nuclease activity. In some embodiments, a Cas protein may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas protein may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al (2015).

In some embodiments, the RNP complex described herein comprises a nickase and a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase Cas is used together with two separate guide RNAs targeting opposite strands of DNA to produce a double nick in the target DNA. In some embodiments, a nickase Cas is used together with two separate guide RNAs that are selected to be in close proximity to produce a double nick in the target DNA.

In some embodiments, chimeric Cas proteins are used, where one domain or region of the protein is replaced by a portion of a different protein. In some embodiments, a Cas nuclease domain may be replaced with a domain from a different nuclease such as Fokl. In some embodiments, a Cas protein may be a modified nuclease.

In other embodiments, the Cas protein may be from a Type-I CRISPR/Cas system. In some embodiments, the Cas protein may be a component of the Cascade complex of a Type-I CRISPR/Cas system. In some embodiments, the Cas protein may be a Cas3 protein. In some embodiments, the Cas protein may be from a Type-III CRISPR/Cas system. In some embodiments, the Cas protein may have an RNA cleavage activity.

### Donor Constructs

As described herein, in some embodiments, the guide RNAs comprising the guide sequences provided herein together with an RNA-guided DNA binding agent (such as a Cas nuclease) induce double-stranded breaks (DSBs) in the HTRA1 gene, and non-homologous ending joining (NHEJ) during repair leads to a mutation in the HTRA1 gene. In some embodiments, NHEJ leads to a deletion or insertion of a nucleotide(s), which induces a frame shift or nonsense mutation in the HTRA1 gene, rendering the gene nonfunctional (e.g., the HTRA1 protein is not expressed). Thus, in some embodiments, the compositions and methods described herein does not include a donor construct.

In some embodiments, the compositions and methods described herein include the use of a nucleic acid construct (“repair template” or “donor template”) that comprises a sequence (a donor/repair sequence) to be inserted into the HTRA1 gene by targeted homology directed repair (HDR). For example, it may be desirable to ensure accurate mutagenesis within the HTRA1 gene to effect a knockout or knockdown of the gene. Methods of designing sequences with appropriate homology arms to generate a desired mutation (e.g., missense or nonsense mutation), or to correct a mutation are known in the art. For example, a stop codon can be inserted at a desired location within the HTRA1 gene. As a further example, the HTRA1 gene can be replaced with another transgene for expression.

In some embodiments, the compositions and methods described herein may be used to alter a polymorphism in 10q26 in a human patient such that HTRA1 expression is reduced. In some embodiments, the polymorphism to be altered is selected from the group consisting of: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In some embodiments, any of the compositions or methods disclosed herein removes the polymorphism and/or replaces the polymorphism with one or more alternative nucleotides. In some embodiments, the compositions and methods described herein may be used to correct a missense mutation or replace a mutant copy of an HTRA1 gene with a wildtype copy of an HTRA1 gene. In some embodiments, the compositions and methods described herein may be used to insert a donor construct that encodes a wildtype HTRA1 protein (e.g, SEQ ID NO: 273). In some embodiments, the one or more mutations to be corrected correspond to a G120D, I179N, A182Profs*33, G206R, A252T, I256T, G276A, G283E, Q289T, P285L, V297M, R302Q, R302X (a stop codon at position 370), T319I, N324T, and R370X as compared to the reference amino acid sequence of SEQ ID NO: 273. In some embodiments, the mutant copy of the HTRA1 gene encompasses any of the following mutations: G120D, I179N, A182Profs*33, G206R, A252T, I256T, G276A, G283E, Q289T, P285L, V297M, R302Q, R302X (a stop codon at position 370), T319I, N324T, and/or R370X as compared to the reference amino acid sequence of SEQ ID NO: 273. In some embodiments, the donor construct comprises a nucleotide sequence that is at least 80%, 85%, 90%, 91%, 92%, 93%, 94%, 95%, 96%, 97%, 98%, 99%, or 100% SEQ ID NO: 272, or a fragment and/or complement thereof.

In some embodiments, the construct is a DNA construct. Methods of designing and making various functional/structural modifications to donor constructs are known in the art. In some embodiments, the construct may comprise any one or more of a polyadenylation tail sequence, a polyadenylation signal sequence, splice acceptor site, or selectable marker. In some embodiments, the polyadenylation tail sequence is encoded, e.g., as a “poly-A” stretch, at the 3′ end of the coding sequence. Methods of designing a suitable polyadenylation tail sequence and/or polyadenylation signal sequence are well known in the art. For example, the polyadenylation signal sequence AAUAAA (SEQ ID NO: 276) is commonly used in mammalian systems, although variants such as UAUAAA (SEQ ID NO: 277) or AU/GUAAA (SEQ ID NO: 278) have been identified. See, e.g., N J Proudfoot, Genes & Dev. 25(17):1770-82, 2011.

The length of the construct can vary, depending on the size of the gene or gene fragment to be inserted, and can be, for example, from 2 base pairs (bp) to 5 bp, from 4 bp to 10 bp, from 5 bp to 20 bp, from 20 bp to 50 bp, from 50 bp to 100 bp, from 100 to 200 bp, from 200 bp to about 5000 bp, such as about 200 bp to about 2000 bp, such as about 500 bp to about 1500 bp. In some embodiments, the length of the DNA donor template is about 200 bp, or is about 500 bp, or is about 800 bp, or is about 1000 base pairs, or is about 1500 base pairs. In other embodiments, the length of the donor template is at least 200 bp, or is at least 500 bp, or is at least 800 bp, or is at least 1000 bp, or is at least 1500 bp, or at least 2000, or at least 2500, or at least 3000, or at least 3500, or at least 4000, or at least 4500, or at least 5000.

The construct can be DNA or RNA, single-stranded, double-stranded or partially single- and partially double-stranded and can be introduced into a host cell in linear or circular (e.g., minicircle) form. See, e.g., U.S. Patent Publication Nos. 2010/0047805, 2011/0281361, 2011/0207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and 0-methyl ribose or deoxyribose residues. A construct can be introduced into a cell as part of a vector molecule having additional sequences such as, for example, replication origins, promoters and genes encoding antibiotic resistance. Moreover, donor constructs can be introduced as naked nucleic acid, as nucleic acid complexed with an agent such as a liposome or poloxamer, or can be delivered by viruses (e.g., adenovirus, AAV, herpesvirus, retrovirus, lentivirus), as described herein.

### Vectors Comprising Guide RNAs

In certain embodiments, the present disclosure provides DNA vectors comprising any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding a RNA-guided DNA binding agent (e.g., Cas9). In some embodiments, the vector comprises a nucleotide sequence encoding a crRNA, a trRNA, or a crRNA and trRNA. In some embodiments, the vector comprises a nucleotide sequence encoding a sgRNA. In some embodiments, the vector comprises a nucleotide sequence encoding a crRNA and an mRNA encoding a Cas protein, such as, Cas9. In some embodiments, the vector comprises a nucleotide sequence encoding a crRNA, a trRNA, and an mRNA encoding a Cas protein, such as, Cas9. In some embodiments, the vector comprises a nucleotide sequence encoding a sgRNA and an mRNA encoding a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

### Delivery of Guide RNA

The guide RNA and RNA-guided DNA binding agents (e.g., Cas nuclease) disclosed herein can be delivered to a host cell or subject, in vivo or ex vivo, using various known and suitable methods available in the art. In some embodiments, a donor construct can also be delivered using various known methods available in the art. The guide RNA, RNA-guided DNA binding agents, and/or donor construct can be delivered individually or together in any combination, using the same or different delivery methods as appropriate.

Conventional viral and non-viral based gene delivery methods can be used to introduce the guide RNA disclosed herein as well as the RNA-guided DNA binding agent and/or donor template in cells (e.g., mammalian cells) and target tissues. As further provided herein, non-viral vector delivery systems nucleic acids such as plasmid vectors, and, e.g., naked nucleic acid, and nucleic acid complexed with a delivery vehicle such as a liposome, lipid nanoparticle (LNP), or poloxamer. Viral vector delivery systems include DNA and RNA viruses.

Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by AmaxaBiosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). The preparation of lipid:nucleic acid complexes, including targeted liposomes such as immunolipid complexes, is well known in the art, and as described herein.

Various delivery systems (e.g., vectors, liposomes, LNPs) containing the guide RNAs, RNA-guided DNA binding agent, and donor construct, singly or in combination, can also be administered to an organism for delivery to cells in vivo or administered to a cell or cell culture ex vivo. Administration is by any of the routes normally used for introducing a molecule into ultimate contact with blood, fluid, or cells including, but not limited to, injection, infusion, topical application and electroporation. In some embodiments, the compositions described herein can be administered intraocularly (e.g., intravitreally or subretinally). Suitable methods of administering such nucleic acids are available and well known to those of skill in the art.

In some embodiments, the guide RNA compositions described herein, alone or encoded on one or more vectors, are formulated in or administered via a lipid nanoparticle; see e.g., WO 2018119182, the contents of which are hereby incorporated by reference in their entirety. Any lipid nanoparticle (LNP) formulation known to those of skill in the art to be capable of delivering nucleotides to subjects may be utilized with the guide RNAs described herein, as well as either mRNA encoding an RNA-guided DNA binding agent such as Cas or Cas9, or an RNA-guided DNA binding agent such as Cas or Cas9 protein itself.

In some embodiments, the present disclosure provides a method for delivering any one of the guide RNAs disclosed herein to a subject, wherein the guide RNA is associated with an LNP. In some embodiments, the guide RNA/LNP is also associated with an RNA-guided DNA binding agent such as Cas9 or an mRNA encoding an RNA-guided DNA binding agent such as Cas9.

In some embodiments, the present disclosure provides a composition comprising any one of the gRNAs disclosed and an LNP. In some embodiments, the composition further comprises a Cas9 or an mRNA encoding Cas9.

In some embodiments, the LNPs comprise cationic lipids. In some embodiments, the LNPs comprise a lipid such as a CCD lipid such as Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate)), Lipid B (((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl) bis(decanoate)), Lipid C (24(44(3-(dimethylamino)propoxy)carbonyl)oxy)hexadecanoyl)oxy)propane-1,3-diyl(9Z,9′Z, 12Z, 12′Z)-bis(octadeca-9, 12-dienoate)), or Lipid D (-(((3-(dimethylamino)propoxy)carbonyl)oxy)-13-(octanoyloxy)tridecyl 3-octylundecanoate). In some embodiments, the LNPs comprise molar ratios of a cationic lipid amine to RNA phosphate (N:P) of about 4.5.

Electroporation is also a well-known means for delivery of cargo, and any electroporation methodology may be used for delivery of any one of the gRNAs disclosed herein. In some embodiments, electroporation may be used to deliver any one of the gRNAs disclosed herein and an RNA-guided DNA binding agent such as Cas9 or an mRNA encoding an RNA-guided DNA binding agent such as Cas9.

In some embodiments, the present disclosure provides a method for delivering any one of the gRNAs disclosed herein to an ex vivo cell, wherein the gRNA is associated with an LNP or not associated with an LNP. In some embodiments, the gRNA/LNP or gRNA is also associated with an RNA-guided DNA binding agent such as Cas9 or an mRNA encoding an RNA-guided DNA agent such as Cas9.

In certain embodiments, the present disclosure comprises DNA or RNA vectors encoding any of the guide RNAs comprising any one or more of the guide sequences described herein. In certain embodiments, the invention comprises DNA or RNA vectors encoding any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA binding agent, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA binding agent, which can be a Cas protein, such as Cas9 or Cpf1. In some embodiments, the vector comprises one or more nucleotide sequence(s) encoding a crRNA, a trRNA, and an mRNA encoding an RNA-guided DNA binding agent, which can be a Cas protein, such as, Cas9 or Cpf1. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the crRNA and the trRNA are encoded by non-contiguous nucleic acids within one vector. In other embodiments, the crRNA and the trRNA may be encoded by a contiguous nucleic acid. In some embodiments, the crRNA and the trRNA are encoded by opposite strands of a single nucleic acid. In other embodiments, the crRNA and the trRNA are encoded by the same strand of a single nucleic acid.

In some embodiments, the vector may be circular. In other embodiments, the vector may be linear. In some embodiments, the vector may be enclosed in a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

In some embodiments, the vector may be a viral vector. In some embodiments, the viral vector may be genetically modified from its wild type counterpart. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some embodiments, a portion of the viral genome may be deleted such that the virus is capable of packaging exogenous sequences having a larger size. In some embodiments, the viral vector may have an enhanced transduction efficiency. In some embodiments, the immune response induced by the virus in a host may be reduced. In some embodiments, viral genes (such as, e.g., integrase) that promote integration of the viral sequence into a host genome may be mutated such that the virus becomes non-integrating. In some embodiments, the viral vector may be replication defective. In some embodiments, the viral vector may comprise exogenous transcriptional or translational control sequences to drive expression of coding sequences on the vector. In some embodiments, the virus may be helper-dependent. For example, the virus may need one or more helper virus to supply viral components (such as, e.g., viral proteins) required to amplify and package the vectors into viral particles. In such a case, one or more helper components, including one or more vectors encoding the viral components, may be introduced into a host cell along with the vector system described herein. In other embodiments, the virus may be helper-free. For example, the virus may be capable of amplifying and packaging the vectors without any helper virus. In some embodiments, the vector system described herein may also encode the viral components required for virus amplification and packaging.

Non-limiting exemplary viral vectors include adeno-associated virus (AAV) vector, lentivirus vectors, adenovirus vectors, helper dependent adenoviral vectors (HDAd), herpes simplex virus (HSV-1) vectors, bacteriophage T4, baculovirus vectors, and retrovirus vectors.

In some embodiments, the viral vector may be an AAV vector. In some embodiments, “AAV” refers all serotypes, subtypes, and naturally-occuring AAV as well as recombinant AAV. “AAV” may be used to refer to the virus itself or a derivative thereof. The term “AAV” includes AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, nonprimate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV). An AAV vector may either be single-stranded (ssAAV) or self-complementary (scAAV).

In other embodiments, the viral vector may a lentivirus vector. In some embodiments, the lentivirus may be non-integrating. In some embodiments, the viral vector may be an adenovirus vector. In some embodiments, the adenovirus may be a high-cloning capacity or “gutless” adenovirus, where all coding viral regions apart from the 5′ and 3′ inverted terminal repeats (ITRs) and the packaging signal (‘I’) are deleted from the virus to increase its packaging capacity. In yet other embodiments, the viral vector may be an HSV-1 vector. In some embodiments, the HSV-1-based vector is helper dependent, and in other embodiments it is helper independent. For example, an amplicon vector that retains only the packaging sequence requires a helper virus with structural components for packaging, while a 30kb-deleted HSV-1 vector that removes non-essential viral functions does not require helper virus. In additional embodiments, the viral vector may be bacteriophage T4. In some embodiments, the bacteriophage T4 may be able to package any linear or circular DNA or RNA molecules when the head of the virus is emptied. In further embodiments, the viral vector may be a baculovirus vector. In yet further embodiments, the viral vector may be a retrovirus vector. In embodiments using AAV or lentiviral vectors, which have smaller cloning capacity, it may be necessary to use more than one vector to deliver all the components of a vector system as disclosed herein. For example, one AAV vector may contain sequences encoding an RNA-guided DNA binding agent such as a Cas protein (e.g., Cas9), while a second AAV vector may contain one or more guide sequences.

In some embodiments, the vector may be capable of driving expression of one or more coding sequences in a cell. In some embodiments, the cell may be a prokaryotic cell, such as, e.g., a bacterial cell. In some embodiments, the cell may be a eukaryotic cell, such as, e.g., a yeast, plant, insect, or mammalian cell. In some embodiments, the eukaryotic cell may be a mammalian cell. In some embodiments, the eukaryotic cell may be a rodent cell. In some embodiments, the eukaryotic cell may be a human cell. Suitable promoters to drive expression in different types of cells are known in the art. In some embodiments, the promoter may be wild type. In other embodiments, the promoter may be modified for more efficient or efficacious expression. In yet other embodiments, the promoter may be truncated yet retain its function. For example, the promoter may have a normal size or a reduced size that is suitable for proper packaging of the vector into a virus.

In some embodiments, the vector may comprise a nucleotide sequence encoding an RNA-guided DNA binding agent such as a Cas protein (e.g., Cas9) described herein. In some embodiments, the nuclease encoded by the vector may be a Cas protein. In some embodiments, the vector system may comprise one copy of the nucleotide sequence encoding the nuclease. In other embodiments, the vector system may comprise more than one copy of the nucleotide sequence encoding the nuclease. In some embodiments, the nucleotide sequence encoding the nuclease may be operably linked to at least one transcriptional or translational control sequence. In some embodiments, the nucleotide sequence encoding the nuclease may be operably linked to at least one promoter.

In some embodiments, the promoter may be constitutive, inducible, or tissue-specific. In some embodiments, the promoter may be a constitutive promoter. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In some embodiments, the promoter may be a tissue-specific promoter, e.g., a promoter specific for expression in specific tissue of the eye.

The vector may further comprise a nucleotide sequence encoding the guide RNA described herein. In some embodiments, the vector comprises one copy of the guide RNA. In other embodiments, the vector comprises more than one copy of the guide RNA. In embodiments with more than one guide RNA, the guide RNAs may be non-identical such that they target different target sequences, or may be identical in that they target the same target sequence. In some embodiments where the vectors comprise more than one guide RNA, each guide RNA may have other different properties, such as activity or stability within a complex with an RNA-guided DNA nuclease, such as a Cas RNP complex. In some embodiments, the nucleotide sequence encoding the guide RNA may be operably linked to at least one transcriptional or translational control sequence, such as a promoter, a 3′ UTR, or a 5′ UTR. In one embodiment, the promoter may be a tRNA promoter, e.g., tRNA^{Lys3}, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters include U6 and H1 promoters. In some embodiments, the nucleotide sequence encoding the guide RNA may be operably linked to a mouse or human U6 promoter. In other embodiments, the nucleotide sequence encoding the guide RNA may be operably linked to a mouse or human H1 promoter. In embodiments with more than one guide RNA, the promoters used to drive expression may be the same or different. In some embodiments, the nucleotide encoding the crRNA of the guide RNA and the nucleotide encoding the trRNA of the guide RNA may be provided on the same vector. In some embodiments, the nucleotide encoding the crRNA and the nucleotide encoding the trRNA may be driven by the same promoter. In some embodiments, the crRNA and trRNA may be transcribed into a single transcript. For example, the crRNA and trRNA may be processed from the single transcript to form a double-molecule guide RNA. Alternatively, the crRNA and trRNA may be transcribed into a single-molecule guide RNA (sgRNA). In other embodiments, the crRNA and the trRNA may be driven by their corresponding promoters on the same vector. In yet other embodiments, the crRNA and the trRNA may be encoded by different vectors.

In some embodiments, the nucleotide sequence encoding the guide RNA may be located on the same vector comprising the nucleotide sequence encoding an RNA-guided DNA binding agent such as a Cas protein. In some embodiments, expression of the guide RNA and of the RNA-guided DNA binding agent such as a Cas protein may be driven by their own corresponding promoters. In some embodiments, expression of the guide RNA may be driven by the same promoter that drives expression of the RNA-guided DNA binding agent such as a Cas protein. In some embodiments, the guide RNA and the RNA-guided DNA binding agent such as a Cas protein transcript may be contained within a single transcript. For example, the guide RNA may be within an untranslated region (UTR) of the RNA-guided DNA binding agent such as a Cas protein transcript. In some embodiments, the guide RNA may be within the 5′ UTR of the transcript. In other embodiments, the guide RNA may be within the 3′ UTR of the transcript. In some embodiments, the intracellular half-life of the transcript may be reduced by containing the guide RNA within its 3′ UTR and thereby shortening the length of its 3′ UTR. In additional embodiments, the guide RNA may be within an intron of the transcript. In some embodiments, suitable splice sites may be added at the intron within which the guide RNA is located such that the guide RNA is properly spliced out of the transcript. In some embodiments, expression of the RNA-guided DNA binding agent such as a Cas protein and the guide RNA from the same vector in close temporal proximity may facilitate more efficient formation of the CRISPR RNP complex.

In some embodiments, the compositions comprise a vector system. In some embodiments, the vector system may comprise one single vector. In other embodiments, the vector system may comprise two vectors. In additional embodiments, the vector system may comprise three vectors. When different guide RNAs are used for multiplexing, or when multiple copies of the guide RNA are used, the vector system may comprise more than three vectors.

In some embodiments, the vector system may comprise inducible promoters to start expression only after it is delivered to a target cell. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech).

In additional embodiments, the vector system may comprise tissue-specific promoters to start expression only after it is delivered into a specific tissue.

The vector may be delivered by liposome, a nanoparticle, an exosome, or a microvesicle. The vector may also be delivered by a lipid nanoparticle (LNP). Any of the LNPs and LNP formulations described herein are suitable for delivery of the guides alone or together a cas nuclease or an mRNA encoding a cas nuclease. In some embodiments, an LNP composition is encompassed comprising: an RNA component and a lipid component, wherein the lipid component comprises an amine lipid, a neutral lipid, a helper lipid, and a stealth lipid; and wherein the N/P ratio is about 1-10.

In some instances, the lipid component comprises Lipid A, cholesterol, DSPC, and PEG-DMG; and wherein the N/P ratio is about 1-10. In some embodiments, the lipid component comprises: about 40-60 mol-% amine lipid; about 5-15 mol-% neutral lipid; and about 1.5-10 mol-% PEG lipid, wherein the remainder of the lipid component is helper lipid, and wherein the N/P ratio of the LNP composition is about 3-10. In some embodiments, the lipid component comprises about 50-60 mol-% amine lipid; about 8-10 mol-% neutral lipid; and about 2.5-4 mol-% PEG lipid, wherein the remainder of the lipid component is helper lipid, and wherein the N/P ratio of the LNP composition is about 3-8. In some instances, the lipid component comprises: about 50-60 mol-% amine lipid; about 5-15 mol-% DSPC; and about 2.5-4 mol-% PEG lipid, wherein the remainder of the lipid component is cholesterol, and wherein the N/P ratio of the LNP composition is about 3-8. In some instances, the lipid component comprises: 48-53 mol-% Lipid A; about 8-10 mol-% DSPC; and 1.5-10 mol-% PEG lipid, wherein the remainder of the lipid component is cholesterol, and wherein the N/P ratio of the LNP composition is 3-8 ±0.2.

In some embodiments, the vector may be delivered systemically. In some embodiments, the vector may be delivered intravitreally or subretinally.

### Pharmaceutical Compositions

Also provided herein are pharmaceutical compositions any of the gRNAs and/or RNA-guided DNA binding agents disclosed herein (or nucleic acids encoding any of the RNA-guided binding agents disclosed herein), and a pharmaceutically acceptable carrier. The pharmaceutical compositions may be suitable for any mode of administration described herein; for example, by intravitreal or intravenous administration.

In some embodiments, use of any of the compositions disclosed herein (e.g., a composition comprising any of the gRNAs and/or RNA-guided DNA binding agents disclosed herein) for treating retinal diseases, such as LCA, retinitis pigmentosa, and age-related macular degeneration require the localized delivery of the composition to the cells in the retina. In some embodiments, the cells that will be the treatment target in these diseases are either the photoreceptor cells in the retina or the cells of the RPE underlying the neurosensory retina.

In some embodiments, the pharmaceutical compositions comprising any of the compositions described herein (e.g., a composition comprising any of the gRNAs and/or RNA-guided DNA binding agents disclosed herein) and a pharmaceutically acceptable carrier are suitable for administration to a human subject. Such carriers are well known in the art (see, e.g., Remington's Pharmaceutical Sciences, 15th Edition, pp. 1035-1038 and 1570-1580). In some embodiments, the pharmaceutical compositions comprising any of the compositions described herein and a pharmaceutically acceptable carrier is suitable for ocular injection. In some embodiments, the pharmaceutical composition is suitable for intravitreal injection. In some embodiments, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, any of the compositions disclosed herein (e.g., a composition comprising any of the gRNAs and/or RNA-guided DNA binding agents disclosed herein) is formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the composition is to be stored long-term, it may be frozen in the presence of glycerol or Tween20. In another embodiment, the pharmaceutically acceptable carrier comprises a surfactant, such as perfluorooctane (Perfluoron liquid).

In certain embodiments of the methods described herein, the pharmaceutical composition described above is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Routes of administration may be combined, if desired.

In some embodiments, any of the pharmaceutical compositions disclosed herein (e.g., a composition comprising any of the gRNAs and RNA-guided DNA binding agents disclosed herein (or nucleic acids encoding any of the gRNAs and RNA-guided DNA binding agents disclosed herein) are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In particular embodiments, the compositions disclosed herein target vascular endothelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration). In some embodiments, intravitreal administration is chosen if the composition is to be administered to an elderly adult (e.g., at least 60 years of age). In particular embodiments, any of the compositions disclosed herein are administered to a subject intravitreally. Procedures for intravitreal injection are known in the art (see, e.g., Peyman, G.A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antiobiotic. The eye may also be rinsed to remove excess sterilizing agent.

Furthermore, in certain embodiments it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

The composition may be delivered in a volume of from about 0.1 μL to about 1 mL, including all numbers within the range, depending on the size of the area to be treated, the amount of the components of the composition (e.g., the amount of gRNA and/or RNA-guided DNA binding agent/nucleotide encoding an RNA-guided DNA binding agent), the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL.

In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

### Methods of Treatment/Prophylaxis

Described herein are various methods of preventing, treating, arresting progression of or ameliorating the ocular disorders and retinal changes associated therewith. Generally, the methods include administering to a mammalian subject in need thereof, an effective amount of a composition comprising any of the guide RNA compositions and RNA-guided DNA binding agent disclosed herein. Any of the guide RNA compositions and RNA-guided DNA binding agent disclosed herein are useful in the methods described below.

In some embodiments, any of the compositions disclosed herein (e.g., a composition comprising any of the gRNAs and RNA-guided DNA binding agents disclosed herein) are for use in treating retinal diseases, such as LCA, retinitis pigmentosa, and age-related macular degeneration may require the localized delivery of the composition to the cells in the retina. The cells that will be the treatment target in these diseases are either the photoreceptor cells in the retina or the cells of the RPE underlying the neurosensory retina. In some embodiments, delivering any of the compositions disclosed herein to these cells requires injection into the subretinal space between the retina and the RPE. In some embodiments, any of the compositions disclosed herein are administered intravitreally or intravenously.

In a certain aspect, the disclosure provides a method of treating a subject having age-related macular degeneration (AMD), comprising the step of administering to the subject any of the compositions disclosed herein (e.g., a composition comprising any of the gRNAs and RNA-guided DNA binding agents disclosed herein). In some embodiments, the AMD is any one of Early AMD; Intermediate AMD; Advanced non-neovascular (“Dry”) AMD; or Advanced neovascular (“Wet”) AMD. In some embodiments, the disclosure provides for methods of treating a subject with Wet AMD. In some embodiments, the disclosure provides for methods of treating a subject with Dry AMD. In some embodiments, the disclosure provides for methods of treating a subject with polyploidal choroidal vasculopathy (PCV). In some embodiments, the subject has geographic atrophy. In some embodiments, the disclosure provides for methods of treating a subject with CARASIL.

In certain embodiments, the pharmaceutical compositions of the disclosure comprise a pharmaceutically acceptable carrier. In certain embodiments, the pharmaceutical compositions of the disclosure comprise PBS. In certain embodiments, the pharmaceutical compositions of the disclosure comprise pluronic. In certain embodiments, the pharmaceutical compositions of the disclosure comprise PBS, NaCl and pluronic.

In some embodiments, the disclosure provides for a method of administering a composition comprising any of the gRNAs disclosed herein to a subject in need thereof. In some embodiments, the method further comprises administering to the subject any of the RNA-guided

DNA binding agents disclosed herein or a polynucleotide encoding any of the RNA-guided DNA binding agents herein. In some embodiments, the gRNA and the RNA-guided binding agent (or polynucleotide encoding the RNA-guided DNA binding agent) are administered to the subject in the same composition. In some embodiments, the gRNA and the RNA-guided binding agent (or polynucleotide encoding the RNA-guided DNA binding agent) are administered to the subject in separate compositions. In some embodiments, the gRNA and the RNA-guided binding agent (or polynucleotide encoding the RNA-guided DNA binding agent) are administered to the subject in separate compositions simultaneously. In some embodiments, the gRNA and the RNA-guided binding agent (or polynucleotide encoding the RNA-guided DNA binding agent) are administered to the subject in separate compositions consecutively (at different times).

In some embodiments, any of the compositions disclosed herein (e.g., a composition comprising any of the gRNAs and RNA-guided DNA binding agents disclosed herein) may be used in a method of knocking down or knocking out HTRA1 gene expression, e.g., in a subject in need thereof. In some embodiments, any of the compositions disclosed herein (e.g., a composition comprising any of the gRNAs and RNA-guided DNA binding agents disclosed herein) is capable of reducing/inhibiting HTRA1 protein expression in a subject in need thereof. In some embodiments, the compositions disclosed herein are capable of reducing/inhibiting HTRA1 expression by at least 5%, 10%, 15%, 25%, 30%, 35%, 40%, 45%, 50%, 55%, 60%, 65%, 70%, 75%, 80%, 85%, 90%, 95% or 100% as compared to the level of HTRA1 protein in a subject in the absence of the composition. In some embodiments, any of the compositions disclosed herein are capable of inhibiting HTRA1 protein expression in a cell by at least 5%, 10%, 15%, 25%, 30%, 35%, 40%, 45%, 50%, 55%, 60%, 65%, 70%, 75%, 80%, 85%, 90%, 95% or 100% as compared to the level of HTRA1 protein expression in the same cell type in the absence of the composition. In some embodiments, any of the compositions disclosed herein is capable of inhibiting HTRA1 protein expression in an eye by at least 5%, 10%, 15%, 25%, 30%, 35%, 40%, 45%, 50%, 55%, 60%, 65%, 70%, 75%, 80%, 85%, 90%, 95% or 100% as compared to the level of HTRA1 protein expression in an eye in the absence of the composition.

In some embodiments, use of any of the compositions (e.g., a composition comprising any of the gRNAs and RNA-guided DNA binding agents disclosed herein) or methods disclosed herein results in a reduction of HTRA1's ability to cleave any one or more HTRA1 substrate in a subject. In some embodiments, the HTRA1 substrate is selected from the group consisting of: fibromodulin, clusterin, ADAMS, elastin, vitronectin, a2-macroglobulin, talin-1, fascin, LTBP-1, EFEMP1, and chloride intracellular channel protein. In some embodiments, use of any of the compositions or methods disclosed herein results in a reduction in HTRA1's ability to cleave a regulator of the complement cascade (e.g., vitronectin, fibromodulin or clusterin). In some embodiments, use of any of the compositions (e.g., a composition comprising any of the gRNAs and RNA-guided DNA binding agents disclosed herein) or methods disclosed herein results in a reduction in HTRA1's ability to cleave an HTRA1 substrate and/or regulator of the complement cascade by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to cleave the HTRA1 substrate and/or regulator of the complement cascade in the absence of the composition or method. In some embodiments, use of any of the compositions or methods disclosed herein results in a reduction in HTRA1's ability to trimerize by at least 5%, 10%, 15%, 25%, 30%, 35%, 40%, 45%, 50%, 55%, 60%, 65%, 70%, 75%, 80%, 85%, 90%, 95% or 100% as compared to the ability of the HTRA1 to trimerize in the absence of the composition or method.

In some embodiments, any of the compositions (e.g., a composition comprising any of the gRNAs and RNA-guided DNA binding agents disclosed herein) and methods described herein may be used to alter a polymorphism in 10q26 in a human patient such that HTRA1 expression is reduced. In some embodiments, the polymorphism to be altered is selected from the group consisting of: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In particular embodiments, the compositions and methods may be used to replace the polymorphism to be altered with one or more polynucleotides. In some embodiments, the method comprises administering a donor construct that replaces the polymorphism in the HTRA1 gene. In some embodiments, the donor construct is administered in the same composition as any of the gRNAs and/or RNA-guided DNA binding agents (or nucleic acids encoding an RNA-guided DNA binding agent). In other embodiments, the donor construct is administered in a separate composition from any of the gRNAs and/or RNA-guided DNA binding agents (or nucleic acids encoding an RNA-guided DNA binding agent).

In some embodiments, any of the compositions (e.g., a composition comprising any of the gRNAs and RNA-guided DNA binding agents disclosed herein) disclosed herein is administered to cell(s) or tissue(s) in a test subject. In some embodiments, the cell(s) or tissue(s) in the test subject express a higher level of HTRA1 than expressed in the same cell type or tissue type in a reference control subject or population of reference control subjects. In some embodiments, the reference control subject is of the same age and/or sex as the test subject. In some embodiments, the reference control subject is a healthy subject, e.g., the subject does not have a disease or disorder of the eye. In some embodiments, the reference control subject does not have a disease or disorder of the eye associated with activation of the complement cascade. In some embodiments, the reference control subject does not have macular degeneration. In some embodiments, the eye or a specific cell type of the eye (e.g., cells in the foveal region) in the test subject express at least 300%, 250%, 200%, 150%, 100%, 95%, 90%, 80%, 70%, 60%, 50%, 40%, 30%, 20%, 10%, 5%, or 1% more HTRA1 as compared to the levels in the reference control subject or population of reference control subjects. In some embodiments, the eye or a specific cell type of the eye (e.g., cells in the foveal region) in the test subject express an HTRA1 gene having any of the mutations disclosed herein. In some embodiments, the eye or a specific cell type of the eye (e.g., cells in the foveal region) in the reference control subject do not express a HTRA1 gene having any of the HTRA1 mutations disclosed herein. In some embodiments, administration any of the guide RNA compositions and RNA-guided DNA binding agent described herein to the cell(s) or tissue(s) of the test subject results in a decrease in levels of HTRA1 protein or functional HTRA1 protein. In some embodiments, administration of any of the guide RNA compositions and RNA-guided DNA binding agent described herein to the cell(s) or tissue(s) of the test subject results in a decrease in levels of HTRA1 protein or functional HTRA1 protein such that the decreased levels are within 90%, 80%, 70%, 60%, 50%, 40%, 30%, 20%, 10%, 5%, or 1% of, or are the same as, the levels of HTRA1 protein or functional HTRA1 protein expressed by the same cell type or tissue type in the reference control subject or population of reference control subjects. In some embodiments, administration of any of the guide RNA compositions and RNA-guided DNA binding agent described herein in the cell(s) or tissue(s) of the test subject results in a decrease in levels of HTRA1 protein or functional HTRA1 protein, but the decreased levels of HTRA1 protein or functional HTRA1 protein are not below the levels of HTRA1 protein or functional HTRA1 protein expressed by the same cell type or tissue type in the reference control subject or population of reference control subjects. In some embodiments, administration of any of the guide RNA compositions and RNA-guided DNA binding agent described herein in the cell(s) or tissue(s) of the test subject results in a decrease in levels of HTRA1 protein or functional HTRA1 protein, but the decreased levels of HTRA1 protein or functional HTRA1 protein are below the levels of HTRA1 protein or functional HTRA1 protein by no more than 1%, 5%, 10%, 20%, 25%, 30%, 40%, 50%, 60%, 70%, 80%, 90% or 100% of the levels expressed by the same cell type or tissue type in the reference control subject or population of reference control subjects.

In some embodiments, any of the treatment and/or prophylactic methods disclosed herein are applied to a subject. In some embodiments, the subject is a mammal. In some embodiments, the subject is a human. In some embodiments, the human is an adult. In some embodiments, the human is an elderly adult. In some embodiments, the human is at least 40, 45, 50, 55, 60, 65, 70, 75, 80, 85, 90, or 95 years of age. In particular embodiments, the human is at least 60 or 65 years of age.

In some embodiments, any of the treatment and/or prophylactic methods disclosed herein is for use in treatment of a patient having one or more mutations that causes macular degeneration (AMD) or that increases the likelihood that a patient develops AMD. In some embodiments, the AMD is Early AMD; Intermediate AMD; Advanced non-neovascular (“Dry”) AMD; or Advanced neovascular (“Wet”) AMD. In some embodiments, the disclosure provides for methods of treating a subject with Wet AMD. In some embodiments, the disclosure provides for methods of treating a subject with Dry AMD. In some embodiments, the disclosure provides for methods of treating a subject with polyploidal choroidal vasculopathy (PCV). In some embodiments, the disclosure provides for methods of treating a subject with CARASIL.

In some embodiments, one or more mutations are in the patient's HTRA1 gene.

In some embodiments, any of the treatment and/or prophylactic methods disclosed herein is for use in treatment of a subject having one or more mutations in the patient's HTRA1 gene. As used herein, “mutations” encompasses polymorphisms that are associated with increased HTRA1 expression. In some embodiments, the one or more mutations result in overexpression of the HTRA1 gene. In some embodiments, HTRA1 is expressed at a level at least 25%, 50%, 75%, 100%, 150%, 200%, 250%, 300%, 350%, 400%, 450%, or 500% greater in the subject having the disease or disorder as compared to the level in a control subject not having the disease or disorder. In some embodiments, the control subject is a subject of the same sex and/or of similar age as the subject having the disease or disorder. In some embodiments, the one or more mutations are not in the coding sequence for the HTRA1 gene. In some embodiments, the one or more mutations are in 10q26 in a human patient. In some embodiments, the one or more mutations correspond to any one or more of the following human polymorphisms: rs61871744; rs59616332; rs11200630; rs61871745; rs11200632; rs11200633; rs61871746; rs61871747; rs370974631; rs200227426; rs201396317; rs199637836; rs11200634; rs75431719; rs10490924; rs144224550; rs36212731; rs36212732; rs36212733; rs3750848; rs3750847; rs3750846; rs566108895; rs3793917; rs3763764; rs11200638; rs1049331; rs2293870; rs2284665; rs60401382; rs11200643; rs58077526; rs932275 and/or rs2142308. In some embodiments, the one or more mutations correspond to a missense mutation. In some embodiments, the missense mutation is a CARASIL-associated mutation. In some embodiments, the one or more mutations correspond to a G120D, I179N, A182Profs*33, G206R, A252T, I256T, G276A, G283E, Q289T, P285L, V297M, R302Q, R302X (a stop codon at position 370), T319I, N324T, and R370X as compared to the reference amino acid sequence of SEQ ID NO: 273.

In some embodiments, the disclosure provides for methods of “correcting” or replacing a mutant HTRA1 gene using any of the compositions disclosed herein, or any combination of those compositions. In some embodiments, the methods are for use in knocking out a portion of a mutant HTRA1 gene and inserting a corresponding wildtype copy of the knocked-out portion of the HTRA1 gene as a donor construct (e.g., a polynucleotide sequence of SEQ ID NO: 272). In some embodiments, the knocked-out portion is the entire HTRA1 gene. In preferred embodiments, the knocked-out portion includes the mutation. In some embodiments, the donor construct is inserted in the same gene locus as the knocked-out portion. In some embodiments, the donor construct is inserted in a different site as the knocked-out portion. In some embodiments, the methods provide for inserting a wildtype copy of the HTRA1 gene (e.g., a polynucleotide having the nucleotide sequence of SEQ ID NO: 272), and not knocking out or replacing the mutant HTRA1 gene.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. As a result, the subject's vision is improved, or vision loss is arrested and/or ameliorated.

In a particular embodiment, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In another embodiment, a method of targeting one or more type(s) of ocular cells for gene augmentation therapy in a subject in need thereof is provided. In another embodiment, a method of targeting one or more type of ocular cells for gene suppression therapy in a subject in need thereof is provided. In yet another embodiment, a method of targeting one or more type of ocular cells for gene knockdown/augmentation therapy in a subject in need thereof is provided. In another embodiment, a method of targeting one or more type of ocular cells for gene correction therapy in a subject in need thereof is provided. In still another embodiment, a method of targeting one or more type of ocular cells for neurotropic factor gene therapy in a subject in need thereof is provided.

In any of the methods described herein, the targeted cell may be an ocular cell. In one embodiment, the targeted cell is a glial cell. In one embodiment, the targeted cell is an RPE cell. In another embodiment, the targeted cell is a photoreceptor. In another embodiment, the photoreceptor is a cone cell. In another embodiment, the targeted cell is a Muller cell. In another embodiment, the targeted cell is a bipolar cell. In yet another embodiment, the targeted cell is a horizontal cell. In another embodiment, the targeted cell is an amacrine cell. In still another embodiment, the targeted cell is a ganglion cell. In still another embodiment, the gene may be expressed and delivered to an intracellular organelle, such as a mitochondrion or a lysosome.

In some embodiments, any of the methods disclosed herein increase photoreceptor function. As used herein “photoreceptor function loss” means a decrease in photoreceptor function as compared to a normal, non-diseased eye or the same eye at an earlier time point. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

In another embodiment, the method includes performing functional and imaging studies to determine the efficacy of the treatment. These studies include ERG and in vivo retinal imaging, as described in the examples below. In addition visual field studies, perimetry and microperimetry, pupillometry, mobility testing, visual acuity, contrast sensitivity, color vision testing may be performed.

In yet another embodiment, any of the above described methods is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod derived cone viability factor 2. The secondary therapy can be administered before, concurrent with, or after administration of any of the compositions described above.

In some embodiments, any of the compositions disclosed herein is administered to a subject in combination with another therapeutic agent or therapeutic procedure. In some embodiments, the additional therapeutic agent is an anti-VEGF therapeutic agent (e.g., such as an anti-VEGF antibody or fragment thereof such as ranibizumab, bevacizumab or aflibercept), a vitamin or mineral (e.g., vitamin C, vitamin E, lutein, zeaxanthin, zinc or copper), omega-3 fatty acids, and/or Visudyne™. In some embodiments, the other therapeutic procedure is a diet having reduced omega-6 fatty acids, laser surgery, laser photocoagulation, submacular surgery, retinal translocation, and/or photodynamic therapy.

### Kits

In some embodiments, any of the compositions disclosed herein (e.g., any of the gRNAs disclosed herein alone or in combination with any of the RNA-guided DNA binding agents disclosed herein) is assembled into a pharmaceutical or diagnostic or research kit to facilitate their use in therapeutic, diagnostic or research applications. A kit may include one or more containers housing any of the compositions disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

### EXAMPLES

The disclosure now being generally described, it will be more readily understood by reference to the following examples, which are included merely for purposes of illustration of certain embodiments and embodiments of the present disclosure, and are not intended to limit the disclosure.

Example 1: Use of gRNA and RNA-guided DNA binding agent for Treating AMD This study will evaluate the efficacy of a composition comprising a gRNA comprising the nucleotide sequence of any one of SEQ ID NOs: 1-271 and an RNA-guided DNA binding agent (e.g., Cas9) for treating patients with AMD. Patients with AMD will be treated with any of these compositions, or a control. The compositions will be administered at varying doses. The compositions will be administered by intravitreal injection in a solution of PBS with additional NaCl and pluronic. Patients will be monitored for improvements in AMD symptoms.

It is expected that treatments with these compositions will improve the AMD symptoms.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

SEQUENCE LISTING
Guide Sequence

| | |
|---|---|
| SEQ ID NO: 1 | UGCGAGUGCGCGCGGCCGCG |
| SEQ ID NO: 2 | GCAGCGGGUGCGAGUGCGCG |
| SEQ ID NO: 3 | AGGAGGGCCUCGGGGGCAGC |
| SEQ ID NO: 4 | CAGGAGGGCCUCGGGGGCAG |
| SEQ ID NO: 5 | ACUCGCACCCGCUGCCCCCG |
| SEQ ID NO: 6 | AGAGUGCAGGAGGGCCUCGG |
| SEQ ID NO: 7 | GAGAGUGCAGGAGGGCCUCG |
| SEQ ID NO: 8 | GGAGAGUGCAGGAGGGCCUC |
| SEQ ID NO: 9 | GGGAGAGUGCAGGAGGGCCU |
| SEQ ID NO: 10 | GCGCCGGGGAGAGUGCAGGA |
| SEQ ID NO: 11 | GGCGCCGGGGAGAGUGCAGG |
| SEQ ID NO: 12 | AGCGGCGCCGGGGAGAGUGC |
| SEQ ID NO: 13 | AGGCCCUCCUGCACUCUCCC |
| SEQ ID NO: 14 | AGGGCCGGAGAGCGGCGCCG |
| SEQ ID NO: 15 | GAGGGCCGGAGAGCGGCGCC |
| SEQ ID NO: 16 | CGAGGGCCGGAGAGCGGCGC |
| SEQ ID NO: 17 | CUCUCCCCGGCGCCGCUCUC |
| SEQ ID NO: 18 | ACAGGGCGAGGGCCGGAGAG |
| SEQ ID NO: 19 | GCGGCGGACAGGGCGAGGGC |
| SEQ ID NO: 20 | GGUGGCGGCGGACAGGGCGA |
| SEQ ID NO: 21 | CGGUGGCGGCGGACAGGGCG |
| SEQ ID NO: 22 | GGCGGCGGUGGCGGCGGACA |
| SEQ ID NO: 23 | CGGCGGCGGUGGCGGCGGAC |
| SEQ ID NO: 24 | GGCGGCGGCGGCGGUGGCGG |
| SEQ ID NO: 25 | UCUGGCGGCGGCGGCGGUGG |
| SEQ ID NO: 26 | GACUCUGGCGGCGGCGGCGG |
| SEQ ID NO: 27 | GGCGACUCUGGCGGCGGCGG |
| SEQ ID NO: 28 | CAUGGCGACUCUGGCGGCGG |
| SEQ ID NO: 29 | CUGCAUGGCGACUCUGGCGG |
| SEQ ID NO: 30 | GAUCUGCAUGGCGACUCUGG |
| SEQ ID NO: 31 | CGGGAUCUGCAUGGCGACUC |
| SEQ ID NO: 32 | AGCGGCGCGCGGGAUCUGCA |
| SEQ ID NO: 33 | GCGGGAGAAGAGCGGCGCGC |
| SEQ ID NO: 34 | AGCGGGAGAAGAGCGGCGCG |
| SEQ ID NO: 35 | CAGCAGCAGCGGGAGAAGAG |
| SEQ ID NO: 36 | CCAGCAGCAGCAGCAGCAGC |
| SEQ ID NO: 37 | GCCAGCAGCAGCAGCAGCAG |
| SEQ ID NO: 38 | CCCGCUGCUGCUGCUGCUGC |
| SEQ ID NO: 39 | GCUGCUGCUGCUGCUGCUGG |
| SEQ ID NO: 40 | GCUGCUGGCGGCGCCCGCCU |
| SEQ ID NO: 41 | CGGGACAGCUGCGCCGAGGC |
| SEQ ID NO: 42 | CCGGGACAGCUGCGCCGAGG |
| SEQ ID NO: 43 | GGCCCGGGACAGCUGCGCCG |
| SEQ ID NO: 44 | CCGCCUCGGCGCAGCUGUCC |
| SEQ ID NO: 45 | CGCCUCGGCGCAGCUGUCCC |
| SEQ ID NO: 46 | UCGGCGCAGCUGUCCCGGGC |
| SEQ ID NO: 47 | AGGCGCCGAGCGGCCGGCCC |
| SEQ ID NO: 48 | AAGGCGCCGAGCGGCCGGCC |
| SEQ ID NO: 49 | GCUGUCCCGGGCCGGCCGCU |
| SEQ ID NO: 50 | GGCCAAAGGCGCCGAGCGGC |
| SEQ ID NO: 51 | CGGCGGCCAAAGGCGCCGAG |
| SEQ ID NO: 52 | GGCCGGCCGCUCGGCGCCUU |
| SEQ ID NO: 53 | UCUGGGCACCCGGCGGCCAA |
| SEQ ID NO: 54 | CGCUCGGCGCCUUUGGCCGC |
| SEQ ID NO: 55 | GCUCGGCGCCUUUGGCCGCC |
| SEQ ID NO: 56 | GCAGCGGUCUGGGCACCCGG |
| SEQ ID NO: 57 | CUCGCAGCGGUCUGGGCACC |
| SEQ ID NO: 58 | GCGCCGGCUCGCAGCGGUCU |
| SEQ ID NO: 59 | CGCGCCGGCUCGCAGCGGUC |
| SEQ ID NO: 60 | GGCAGCGCGCCGGCUCGCAG |
| SEQ ID NO: 61 | GUGCCCAGACCGCUGCGAGC |
| SEQ ID NO: 62 | GGCUGCGGCGGGCAGCGCGC |
| SEQ ID NO: 63 | CGCAGUGCUCCGGCUGCGGC |
| SEQ ID NO: 64 | UCGCAGUGCUCCGGCUGCGG |
| SEQ ID NO: 65 | GGCGCGCUGCCCGCCGCAGC |
| SEQ ID NO: 66 | CCCUCGCAGUGCUCCGGCUG |
| SEQ ID NO: 67 | CGGCCGCCCUCGCAGUGCUC |
| SEQ ID NO: 68 | GCCGCAGCCGGAGCACUGCG |
| SEQ ID NO: 69 | CCGCAGCCGGAGCACUGCGA |
| SEQ ID NO: 70 | CAGCCGGAGCACUGCGAGGG |
| SEQ ID NO: 71 | CGGAGCACUGCGAGGGCGGC |
| SEQ ID NO: 72 | GGAGCACUGCGAGGGCGGCC |
| SEQ ID NO: 73 | ACUGCGAGGGCGGCCGGGCC |
| SEQ ID NO: 74 | CUGCGAGGGCGGCCGGGCCC |
| SEQ ID NO: 75 | AGCCGCACGCGUCCCGGGCC |
| SEQ ID NO: 76 | GCAGCAGCCGCACGCGUCCC |
| SEQ ID NO: 77 | CGCAGCAGCCGCACGCGUCC |
| SEQ ID NO: 78 | GGCCGGGCCCGGGACGCGUG |
| SEQ ID NO: 79 | GGACGCGUGCGGCUGCUGCG |
| SEQ ID NO: 80 | UGCGGCUGCUGCGAGGUGUG |
| SEQ ID NO: 81 | CGAGGUGUGCGGCGCGCCCG |
| SEQ ID NO: 82 | GAGGUGUGCGGCGCGCCCGA |
| SEQ ID NO: 83 | AGGCCGCACGCGGCGCCCUC |
| SEQ ID NO: 84 | CAGGCCGCACGCGGCGCCCU |
| SEQ ID NO: 85 | GCGCCCGAGGGCGCCGCGUG |
| SEQ ID NO: 86 | GCCCUCCUGCAGGCCGCACG |
| SEQ ID NO: 87 | GGGCGCCGCGUGCGGCCUGC |
| SEQ ID NO: 88 | CGCCGCGUGCGGCCUGCAGG |
| SEQ ID NO: 89 | GCCGCGUGCGGCCUGCAGGA |
| SEQ ID NO: 90 | CGCCGCACGGGCCCUCCUGC |
| SEQ ID NO: 91 | GGCCUGCAGGAGGGCCCGUG |
| SEQ ID NO: 92 | ACUGCAGCCCCUCGCCGCAC |
| SEQ ID NO: 93 | CACUGCAGCCCCUCGCCGCA |
| SEQ ID NO: 94 | GCAGGAGGGCCCGUGCGGCG |
| SEQ ID NO: 95 | CAGGAGGGCCCGUGCGGCGA |
| SEQ ID NO: 96 | AGGAGGGCCCGUGCGGCGAG |
| SEQ ID NO: 97 | CGGCGAGGGGCUGCAGUGCG |
| SEQ ID NO: 98 | CUGCAGUGCGUGGUGCCCUU |
| SEQ ID NO: 99 | UGCAGUGCGUGGUGCCCUUC |
| SEQ ID NO: 100 | GCAGUGCGUGGUGCCCUUCG |
| SEQ ID NO: 101 | GCCGAGGCUGGCACCCCGAA |
| SEQ ID NO: 102 | GGCCGAGGCUGGCACCCCGA |
| SEQ ID NO: 103 | GCCCUUCGGGGUGCCAGCCU |
| SEQ ID NO: 104 | CGCCGCACCGUGGCCGAGGC |
| SEQ ID NO: 105 | CGGGGUGCCAGCCUCGGCCA |
| SEQ ID NO: 106 | GCGCCGCCGCACCGUGGCCG |
| SEQ ID NO: 107 | UGCCAGCCUCGGCCACGGUG |
| SEQ ID NO: 108 | CUGCGCGCGCCGCCGCACCG |
| SEQ ID NO: 109 | CAGCCUCGGCCACGGUGCGG |
| SEQ ID NO: 110 | CACGGUGCGGCGGCGCGCGC |
| SEQ ID NO: 111 | GUGCGGCGGCGCGCGCAGGC |
| SEQ ID NO: 112 | GCUGGCGCACACACAGAGGC |
| SEQ ID NO: 113 | CGCUGCUGGCGCACACACAG |
| SEQ ID NO: 114 | GCCGCACACCGGCUCGCUGC |
| SEQ ID NO: 115 | UGUGUGCGCCAGCAGCGAGC |
| SEQ ID NO: 116 | GCCAGCAGCGAGCCGGUGUG |
| SEQ ID NO: 117 | UUGGCGUCGCUGCCGCACAC |
| SEQ ID NO: 118 | GCACAGGUUGGCGUAGGUGU |
| SEQ ID NO: 119 | CAGCUGGCACAGGUUGGCGU |
| SEQ ID NO: 120 | GGCGCGCAGCUGGCACAGGU |
| SEQ ID NO: 121 | UGGCGGCGCGCAGCUGGCAC |
| SEQ ID NO: 122 | GGCGGCUGGCGGCGCGCAGC |
| SEQ ID NO: 123 | CCUCUCGGAGCGGCGGCUGG |
| SEQ ID NO: 124 | CAGCCUCUCGGAGCGGCGGC |
| SEQ ID NO: 125 | GGUGCAGCCUCUCGGAGCGG |
| SEQ ID NO: 126 | GCCGGUGCAGCCUCUCGGAG |
| SEQ ID NO: 127 | CCGCCAGCCGCCGCUCCGAG |
| SEQ ID NO: 128 | CGGCGGCCGGUGCAGCCUCU |
| SEQ ID NO: 129 | GCCGCUCCGAGAGGCUGCAC |
| SEQ ID NO: 130 | CGAGAGGCUGCACCGGCCGC |
| SEQ ID NO: 131 | GCAGGACGAUGACCGGCGGC |
| SEQ ID NO: 132 | CGCUGCAGGACGAUGACCGG |
| SEQ ID NO: 133 | CCGCGCUGCAGGACGAUGAC |
| SEQ ID NO: 134 | CCGGUCAUCGUCCUGCAGCG |
| SEQ ID NO: 135 | GGCCGCAGGCUCCGCGCUGC |
| SEQ ID NO: 136 | GUCCUGCAGCGCGGAGCCUG |
| SEQ ID NO: 137 | AUCUUCCUGCCCUUGGCCGC |
| SEQ ID NO: 138 | CAGCGCGGAGCCUGCGGCCA |
| SEQ ID NO: 139 | AGCGCGGAGCCUGCGGCCAA |
| SEQ ID NO: 140 | CGGAGCCUGCGGCCAAGGGC |
| SEQ ID NO: 141 | UGUUGGGAUCUUCCUGCCCU |
| SEQ ID NO: 142 | UAUUUAUGGCGCAAACUGUU |
| SEQ ID NO: 143 | AUAUUUAUGGCGCAAACUGU |
| SEQ ID NO: 144 | CCGCGAUAAAGUUAUAUUUA |
| SEQ ID NO: 145 | CCAUAAAUAUAACUUUAUCG |
| SEQ ID NO: 146 | AUAUAACUUUAUCGCGGACG |
| SEQ ID NO: 147 | UAACUUUAUCGCGGACGUGG |
| SEQ ID NO: 148 | GGAGAAGAUCGCCCCUGCCG |
| SEQ ID NO: 149 | UUCGAUAUGAACCACGGCAG |
| SEQ ID NO: 150 | AUUCGAUAUGAACCACGGCA |
| SEQ ID NO: 151 | AAUUCGAUAUGAACCACGGC |
| SEQ ID NO: 152 | AAACAAUUCGAUAUGAACCA |
| SEQ ID NO: 153 | GGCACCUCUCGUUUAGAAAA |
| SEQ ID NO: 154 | GCUUCCGUUUUCUAAACGAG |
| SEQ ID NO: 155 | GUUUUCUAAACGAGAGGUGC |
| SEQ ID NO: 156 | UUCUAAACGAGAGGUGCCGG |
| SEQ ID NO: 157 | AACCCAGACCCACUAGCCAC |
| SEQ ID NO: 158 | CGAGAGGUGCCGGUGGCUAG |
| SEQ ID NO: 159 | GAGAGGUGCCGGUGGCUAGU |
| SEQ ID NO: 160 | GUGCCGGUGGCUAGUGGGUC |
| SEQ ID NO: 161 | UGCCGGUGGCUAGUGGGUCU |
| SEQ ID NO: 162 | UGGGUCUGGGUUUAUUGUGU |
| SEQ ID NO: 163 | GGGUUUAUUGUGUCGGAAGA |
| SEQ ID NO: 164 | GAUCGUGACAAAUGCCCACG |
| SEQ ID NO: 165 | GUGCUUGUUGGUCACCACGU |
| SEQ ID NO: 166 | GGUGCUUGUUGGUCACCACG |
| SEQ ID NO: 167 | AACUUUGACCCGGUGCUUGU |
| SEQ ID NO: 168 | ACGUGGUGACCAACAAGCAC |
| SEQ ID NO: 169 | CGUGGUGACCAACAAGCACC |
| SEQ ID NO: 170 | UCUUCAGCUCAACUUUGACC |
| SEQ ID NO: 171 | GUCAAAGUUGAGCUGAAGAA |
| SEQ ID NO: 172 | CUUGAUUUUGGCUUCGUAAG |
| SEQ ID NO: 173 | CACUUACGAAGCCAAAAUCA |
| SEQ ID NO: 174 | CUCAUCCACAUCCUUGAUUU |
| SEQ ID NO: 175 | CGAAGCCAAAAUCAAGGAUG |
| SEQ ID NO: 176 | ACUCAUCAAAAUUGACCACC |
| SEQ ID NO: 177 | CUCAUCAAAAUUGACCACCA |
| SEQ ID NO: 178 | GGACAGGCAGCUUGCCCUGG |
| SEQ ID NO: 179 | GCAGGACAGGCAGCUUGCCC |
| SEQ ID NO: 180 | GAGCGGCCAAGCAGCAGGAC |
| SEQ ID NO: 181 | AAGCUGCCUGUCCUGCUGCU |
| SEQ ID NO: 182 | CUGAGGAGCGGCCAAGCAGC |
| SEQ ID NO: 183 | CCGGCCGCAGCUCUGAGGAG |
| SEQ ID NO: 184 | CUCUCCCGGCCGCAGCUCUG |
| SEQ ID NO: 185 | UUGGCCGCUCCUCAGAGCUG |
| SEQ ID NO: 186 | CCGCUCCUCAGAGCUGCGGC |
| SEQ ID NO: 187 | CGCUCCUCAGAGCUGCGGCC |
| SEQ ID NO: 188 | AUGGCGACCACGAACUCUCC |
| SEQ ID NO: 189 | GCUGCGGCCGGGAGAGUUCG |
| SEQ ID NO: 190 | GGAGAGUUCGUGGUCGCCAU |
| SEQ ID NO: 191 | AAGGGAAAACGGGCUUCCGA |
| SEQ ID NO: 192 | CUGUGUUUUGAAGGGAAAAC |
| SEQ ID NO: 193 | ACUGUGUUUUGAAGGGAAAA |
| SEQ ID NO: 194 | GGUGGUGACUGUGUUUUGAA |
| SEQ ID NO: 195 | CGGUGGUGACUGUGUUUUGA |
| SEQ ID NO: 196 | CUUCAAAACACAGUCACCAC |
| SEQ ID NO: 197 | UUCAAAACACAGUCACCACC |
| SEQ ID NO: 198 | GGUGGUGCUCACGAUCCCGG |
| SEQ ID NO: 199 | CUGGGUGGUGCUCACGAUCC |
| SEQ ID NO: 200 | CUCUUUGCCGCCUCGCUGGG |
| SEQ ID NO: 201 | AUCGUGAGCACCACCCAGCG |
| SEQ ID NO: 202 | CAGCUCUUUGCCGCCUCGCU |
| SEQ ID NO: 203 | GUGAGCACCACCCAGCGAGG |
| SEQ ID NO: 204 | CCAGCUCUUUGCCGCCUCGC |
| SEQ ID NO: 205 | CCAGCGAGGCGGCAAAGAGC |
| SEQ ID NO: 206 | CAGCGAGGCGGCAAAGAGCU |
| SEQ ID NO: 207 | AGCGAGGCGGCAAAGAGCUG |
| SEQ ID NO: 208 | UGUAGUCCAUGUCUGAGUUG |
| SEQ ID NO: 209 | GGGGCUCCGCAACUCAGACA |
| SEQ ID NO: 210 | AGUUGAUGAUGGCGUCGGUC |
| SEQ ID NO: 211 | UCCAUAGUUGAUGAUGGCGU |
| SEQ ID NO: 212 | CGAGUUUCCAUAGUUGAUGA |
| SEQ ID NO: 213 | ACCGACGCCAUCAUCAACUA |
| SEQ ID NO: 214 | CAUCAUCAACUAUGGAAACU |
| SEQ ID NO: 215 | AUCAUCAACUAUGGAAACUC |
| SEQ ID NO: 216 | AUCAACUAUGGAAACUCGGG |
| SEQ ID NO: 217 | CACCGUCCAGGUUUACUAAC |
| SEQ ID NO: 218 | UCACCGUCCAGGUUUACUAA |
| SEQ ID NO: 219 | GGGAGGCCCGUUAGUAAACC |
| SEQ ID NO: 220 | GGCCCGUUAGUAAACCUGGA |
| SEQ ID NO: 221 | UUCCAAUCACUUCACCGUCC |
| SEQ ID NO: 222 | AACCUGGACGGUGAAGUGAU |
| SEQ ID NO: 223 | AACACUUUGAAAGUGACAGC |
| SEQ ID NO: 224 | CUUAUCAGAUGGGAUUGCAA |
| SEQ ID NO: 225 | ACUUUUUAAUCUUAUCAGAU |
| SEQ ID NO: 226 | AACUUUUUAAUCUUAUCAGA |
| SEQ ID NO: 227 | UAAGAUUAAAAAGUUCCUCA |
| SEQ ID NO: 228 | GUCGGUCAUGGGACUCCGUG |
| SEQ ID NO: 229 | UCCUUUGGCCUGUCGGUCAU |
| SEQ ID NO: 230 | UUCCUUUGGCCUGUCGGUCA |
| SEQ ID NO: 231 | CACGGAGUCCCAUGACCGAC |
| SEQ ID NO: 232 | UGGCUUUUCCUUUGGCCUGU |
| SEQ ID NO: 233 | UCCCAUGACCGACAGGCCAA |
| SEQ ID NO: 234 | CUUGGUGAUGGCUUUUCCUU |
| SEQ ID NO: 235 | AAUAUACUUCUUCUUGGUGA |
| SEQ ID NO: 236 | GAUACCAAUAUACUUCUUCU |
| SEQ ID NO: 237 | AUCACCAAGAAGAAGUAUAU |
| SEQ ID NO: 238 | UGGACGUGAGUGACAUCAUU |
| SEQ ID NO: 239 | CUUCAGCUCUUUGGCUUUGC |
| SEQ ID NO: 240 | GUGCCGGUCCUUCAGCUCUU |
| SEQ ID NO: 241 | CAGCAAAGCCAAAGAGCUGA |
| SEQ ID NO: 242 | AAGCCAAAGAGCUGAAGGAC |
| SEQ ID NO: 243 | AAGAGCUGAAGGACCGGCAC |
| SEQ ID NO: 244 | AGAGCUGAAGGACCGGCACC |
| SEQ ID NO: 245 | CGUCUGGGAAGUCCCGGUGC |
| SEQ ID NO: 246 | AGAUCACGUCUGGGAAGUCC |
| SEQ ID NO: 247 | ACGCUCCUGAGAUCACGUCU |
| SEQ ID NO: 248 | UACGCUCCUGAGAUCACGUC |
| SEQ ID NO: 249 | GACUUCCCAGACGUGAUCUC |
| SEQ ID NO: 250 | CCAGCUUCUGCUGGGGUAUC |
| SEQ ID NO: 251 | GAGACCACCAGCUUCUGCUG |
| SEQ ID NO: 252 | UGAGACCACCAGCUUCUGCU |
| SEQ ID NO: 253 | UUGAGACCACCAGCUUCUGC |
| SEQ ID NO: 254 | CCUGAUACCCCAGCAGAAGC |
| SEQ ID NO: 255 | GAUACCCCAGCAGAAGCUGG |
| SEQ ID NO: 256 | AGCAGAAGCUGGUGGUCUCA |
| SEQ ID NO: 257 | GACGUCAUAAUCAGCAUCAA |
| SEQ ID NO: 258 | CAGCAUCAAUGGACAGUCCG |
| SEQ ID NO: 259 | GACAUCAUUGGCGGAGACCA |
| SEQ ID NO: 260 | GACGUCGCUGACAUCAUUGG |
| SEQ ID NO: 261 | AAUGACGUCGCUGACAUCAU |
| SEQ ID NO: 262 | AUGUCAGCGACGUCAUUAAA |
| SEQ ID NO: 263 | UGUCAGCGACGUCAUUAAAA |
| SEQ ID NO: 264 | AAGGGAAAGCACCCUGAACA |
| SEQ ID NO: 265 | CCUGCGGACCACCAUGUUCA |
| SEQ ID NO: 266 | CCCUGCGGACCACCAUGUUC |
| SEQ ID NO: 267 | GGAAAGCACCCUGAACAUGG |
| SEQ ID NO: 268 | CCCUGAACAUGGUGGUCCGC |
| SEQ ID NO: 269 | CCUGAACAUGGUGGUCCGCA |
| SEQ ID NO: 270 | CUGAACAUGGUGGUCCGCAG |
| SEQ ID NO: 271 | UGAUAUCUUCAUUACCCCUG |

SEQ ID NO: 272—Human HTRA1 Polynucleotide
Sequence- GenBank Accession No. NM_002775.4
CAATGGGCTGGGCCGCGCGGCCGCGCGCACTCGCACCCGCTGCCCCCG
AGGCCCTCCTGCACTCTCCCCGGCGCCGCTCTCCGGCCCTCGCCCTGT
CCGCCGCCACCGCCGCCGCCGCCAGAGTCGCCATGCAGATCCCGCGCG
CCGCTCTTCTCCCGCTGCTGCTGCTGCTGCTGGCGGCGCCCGCCTCGG
CGCAGCTGTCCCGGGCCGGCCGCTCGGCGCCTTTGGCCGCCGGGTGCC
CAGACCGCTGCGAGCCGGCGCGCTGCCCGCCGCAGCCGGAGCACTGCG
AGGGCGGCCGGGCCCGGGACGCGTGCGGCTGCTGCGAGGTGTGCGGCG
CGCCCGAGGGCGCCGCGTGCGGCCTGCAGGAGGGCCCGTGCGGCGAGG
GGCTGCAGTGCGTGGTGCCCTTCGGGGTGCCAGCCTCGGCCACGGTGC
GGCGGCGCGCGCAGGCCGGCCTCTGTGTGTGCGCCAGCAGCGAGCCGG
TGTGCGGCAGCGACGCCAACACCTACGCCAACCTGTGCCAGCTGCGCG
CCGCCAGCCGCCGCTCCGAGAGGCTGCACCGGCCGCCGGTCATCGTCC
TGCAGCGCGGAGCCTGCGGCCAAGGGCAGGAAGATCCCAACAGTTTGC
GCCATAAATATAACTTTATCGCGGACGTGGTGGAGAAGATCGCCCCTG
CCGTGGTTCATATCGAATTGTTTCGCAAGCTTCCGTTTTCTAAACGAG
AGGTGCCGGTGGCTAGTGGGTCTGGGTTTATTGTGTCGGAAGATGGAC
TGATCGTGACAAATGCCCACGTGGTGACCAACAAGCACCGGGTCAAAG
TTGAGCTGAAGAACGGTGCCACTTACGAAGCCAAAATCAAGGATGTGG
ATGAGAAAGCAGACATCGCACTCATCAAAATTGACCACCAGGGCAAGC
TGCCTGTCCTGCTGCTTGGCCGCTCCTCAGAGCTGCGGCCGGGAGAGT
TCGTGGTCGCCATCGGAAGCCCGTTTTCCCTTCAAAACACAGTCACCA
CCGGGATCGTGAGCACCACCCAGCGAGGCGGCAAAGAGCTGGGGCTCC
GCAACTCAGACATGGACTACATCCAGACCGACGCCATCATCAACTATG
GAAACTCGGGAGGCCCGTTAGTAAACCTGGACGGTGAAGTGATTGGAA
TTAACACTTTGAAAGTGACAGCTGGAATCTCCTTTGCAATCCCATCTG
ATAAGATTAAAAAGTTCCTCACGGAGTCCCATGACCGACAGGCCAAAG
GAAAAGCCATCACCAAGAAGAAGTATATTGGTATCCGAATGATGTCAC
TCACGTCCAGCAAAGCCAAAGAGCTGAAGGACCGGCACCGGGACTTCC
CAGACGTGATCTCAGGAGCGTATATAATTGAAGTAATTCCTGATACCC
CAGCAGAAGCTGGTGGTCTCAAGGAAAACGACGTCATAATCAGCATCA
ATGGACAGTCCGTGGTCTCCGCCAATGATGTCAGCGACGTCATTAAAA
GGGAAAGCACCCTGAACATGGTGGTCCGCAGGGGTAATGAAGATATCA
TGATCACAGTGATTCCCGAAGAAATTGACCCATAGGCAGAGGCATGAG
CTGGACTTCATGTTTCCCTCAAAGACTCTCCCGTGGATGACGGATGAG
GACTCTGGGCTGCTGGAATAGGACACTCAAGACTTTTGACTGCCATTT
TGTTTGTTCAGTGGAGACTCCCTGGCCAACAGAATCCTTCTTGATAGT
TTGCAGGCAAAACAAATGTAATGTTGCAGATCCGCAGGCAGAAGCTCT
GCCCTTCTGTATCCTATGTATGCAGTGTGCTTTTTCTTGCCAGCTTGG
GCCATTCTTGCTTAGACAGTCAGCATTTGTCTCCTCCTTTAACTGAGT
CATCATCTTAGTCCAACTAATGCAGTCGATACAATGCGTAGATAGAAG
AAGCCCCACGGGAGCCAGGATGGGACTGGTCGTGTTTGTGCTTTTCTC
CAAGTCAGCACCCAAAGGTCAATGCACAGAGACCCCGGGTGGGTGAGC
GCTGGCTTCTCAAACGGCCGAAGTTGCCTCTTTTAGGAATCTCTTTGG
AATTGGGAGCACGATGACTCTGAGTTTGAGCTATTAAAGTACTTCTTA
CACATTGCAAAAAAAAAAAAAAAAAA
SEQ ID NO: 273—Human HTRA1 Amino Acid
Sequence- GenBank Accession No. NP_002766.1
MQIPRAALLPLLLLLLAAPASAQLSRAGRSAPLAAGCPDRCEPARCPP
QPEHCEGGRARDACGCCEVCGAPEGAACGLQEGPCGEGLQCVVPFGVP
ASATVRRRAQAGLCVCASSEPVCGSDANTYANLCQLRAASRRSERLHR
PPVIVLQRGACGQGQEDPNSLRHKYNFIADVVEKIAPAVVHIELFRKL
PFSKREVPVASGSGFIVSEDGLIVTNAHVVTNKHRVKVELKNGATYEA
KIKDVDEKADIALIKIDHQGKLPVLLLGRSSELRPGEFVVAIGSPFSL
QNTVTTGIVSTTQRGGKELGLRNSDMDYIQTDAIINYGNSGGPLVNLD
GEVIGINTLKVTAGISFAIPSDKIKKFLTESHDRQAKGKAITKKKYIG
IRMMSLTSSKAKELKDRHRDFPDVISGAYBEVIPDTPAEAGGLKENDV
IISINGQSVVSANDVSDVIKRESTLNMVVRRGNEDIMITVIPEEIDP.

1. A composition comprising a guide RNA and a pharmaceutically acceptable carrier, wherein the guide RNA targets an HTRA1 gene, and wherein the guide RNA comprises a nucleotide sequence that is 100% or at least 99%, 98%, 97%, 96%, 95%, 94%, 93%, 92%, 91%, 90%, 88%, or 85% identical to a sequence selected from SEQ ID NOs: 1-271.

2-3. (canceled)

4. The composition of claim 1, wherein the composition is substantially pyrogen free.

5. The composition of claim 1, wherein the composition further comprises an RNA-guided DNA binding agent or a nucleic acid encoding an RNA-guided DNA binding agent.

6. The composition of claim 5, wherein the RNA-guided DNA binding agent is a Cas protein.

7. The composition of claim 6, wherein the Cas protein is Cas9 or Cpf1.

8. The composition of claim 7, wherein the Cas protein is Cas9 from Streptococcus pyogenes.

9. The composition of claim 1, wherein the composition further comprises a trRNA.

10. The composition of claim 1, wherein the guide RNA further comprises a trRNA.

11. The composition of claim 1, wherein the guide RNA is in a viral vector or a non-viral vector.

12-14. (canceled)

15. The composition of claim 1, wherein the guide RNA comprises a 2′-O-methyl (2′-O-Me) modified nucleotide.

16. The composition of claim 1, wherein the guide RNA comprises a phosphorothioate (PS) bond between nucleotides.

17. A method of inducing a double-stranded break (DSB) within the HTRA1 gene, comprising delivering the composition of claim 1 to a cell.

18. A method of modifying the HTRA1 gene comprising delivering a composition to a cell, the method comprising administering to the cell the composition of claim 5.

19. A method of treating a disease or disorder in a subject in need thereof, wherein the disease or disorder is associated with aberrantly expressed HTRA1, wherein the method comprises administering to the subject the composition of claim 5.

20. The method of claim 19, wherein HTRA1 is expressed at a level at least 25%, 50%, 75%, 100%, 150%, 200%, 250%, 300%, 350%, 400%, 450%, or 500% greater in the subject having the disease or disorder as compared to the level in a control subject not having the disease or disorder.

21. The method of claim 19, wherein the disease or disorder is age-related macular degeneration or polypoidal choroidal vasculopathy.

22-42. (canceled)

43. The method of claim 18, wherein the guide RNA is single-stranded or double-stranded.

44. The method of claim 18, wherein the nucleic acid construct is a single-stranded DNA or a double-stranded DNA.

45. (canceled)

46. The method of claim 19, wherein the subject has one or more mutations in the HTRA1 gene.

47. The method of claim 46, wherein the one or more mutations are not in the coding sequence for the HTRA1 gene or wherein the one or more mutations are in 10q26 in a human subject.

48-61. (canceled)
